(19) United States
(12) Patent Application Publication
(10) Pub. No.: US 2024/0123047 A1
(43) Pub. Date: Apr. 18, 2024

(54) LIPID NANOPARTICLE ADJUVANT COMPOSITION FOR PNEUMOCOCCAL CONJUGATE VACCINES

(71) Applicant: Merck Sharp & Dohme LLC, Rahway, NJ (US)

(72) Inventors: Julie M. Skinner, Phoenixville, PA (US); William J. Smith, Harleysville, PA (US)

(73) Assignee: Merck Sharp & Dohme LLC, Rahway, NJ (US)

(21) Appl. No.: 18/263,277

(22) PCT Filed: Feb. 2, 2022
(86) PCT No.: PCT/US2022/014810

§ 371 Date: Jul. 27, 2023

Related U.S. Application Data
(60) Provisional application No. 63/145,653, filed on Feb. 4, 2021.

(51) Int. Cl. A61K 39/09; A61K 47/64

(52) U.S. Cl. CPC A61K 39/092; A61K 47/646; A61K 2039/55555

(57) ABSTRACT

The present invention relates generally to the prevention of pneumococcal disease. More specifically, the invention relates to compositions comprising pneumococcal conjugates and a lipid nanoparticle (LNP).

### BACKGROUND OF THE INVENTION

Pneumococcal disease is an infection caused by the bacteria Streptococcus pneumoniae (pneumococcus). Different pneumococcal serotypes are known to cause different manifestations of the disease and infections can cause a range of symptoms from ear and sinus infections to pneumonia and bloodstream infections. Pneumococcal disease has a high associated morbidity and mortality worldwide, particularly among the elderly and young children. Currently 100 capsular polysaccharides have been identified (Ganaie, F. et al. (2020) Clinical Science and Epidemiology, Vol. 11, Issue 3, pages 1-15). These serotypes are distinguished by their chemical structure, serological response, and other related genetic mutations.

In 1983, PNEUMOVAX® (Merck Sharp & Dohme Corp., a subsidiary of Merck & Co., Inc., Kenilworth, NJ, USA), a 23-valent pneumococcal vaccine comprising pneumococcal capsular polysaccharides was approved in the United States. This vaccine demonstrated reduced immunogenicity in infants due to T-cell independent responses. To address this issue, particularly in infants, pneumococcal conjugate vaccines (PCVs) were developed by covalently coupling the polysaccharide to a carrier protein, wherein the immunogenic response became T-cell dependent. In 2000, PREVNAR® (Wyeth Pharmaceuticals LLC), a 7-valent pneumococcal vaccine was approved in the United States and in 2010, PREVNAR13®, a 13-valent pneumococcal vaccine was approved in the United States. Other multivalent PCVs are known and licensed worldwide.

Lipid nanoparticles (LNPs) comprising cationic lipids are known in the art. LNPs initially were developed, among other reasons, as delivery vehicles for nucleic acids. See U.S. Pat. No. 7,691,405, US2006/0083780, US2006/0240554, US2008/0020058, US2009/0263407, US2009/0285881, WO2009/086558, WO2009/127060, WO2009/132131, WO2010/042877, WO2010/054384, WO2010/054401, WO2010/054405 and WO2010/054406. Further research has demonstrated that LNPs comprising cationic lipids may be useful as vaccine adjuvants (WO2015/130584).

Licensed PCVs currently utilize aluminum containing derivatives as adjuvants to increase immunogenicity. Even though aluminum adjuvants increase immunogenic responses from baseline, it is unknown whether the immunogenic response is sufficient for higher valency PCVs, particularly in infants. Therefore, there is a need to identify other adjuvants that can provide increased immunogenicity for multivalent PCVs over the current aluminum adjuvant standard(s).

### SUMMARY OF THE INVENTION

The present invention relates generally to the prevention of pneumococcal disease. More specifically, the invention relates to compositions administered as a vaccine which include pneumococcal conjugates and a lipid nanoparticle (LNP). The present disclosure provides, among other things, a pneumococcal conjugate composition including an LNP comprising the cationic lipid: 13Z,16Z)-N,N-dimethyl-3-nonyldocosa-13,16-dien-1-amine (referred to as “Cationic Lipid A” or “CLA”). Further, the present disclosure provides a pneumococcal conjugate composition including an LNP comprising 4 main components: 1) a cationic lipid; 2) a neutral lipid, 3) a phospholipid, and 4) a PEG-lipid. Further, the present disclosure provides a pneumococcal conjugate composition including an LNP comprising 4 main components: 1) (13Z,16Z)-N,N-dimethyl-3-nonyldocosa-13,16-dien-1-amine (CLA); 2) cholesterol; 3) distearoyl phosphatidyl choline (DSPC), and 4) ePEG2000-DMG. Compared to the performance of a) a non-adjuvanted PCV composition; b) an aluminum phosphate adjuvanted (APA) PCV composition; and c) an LNP (comprising other cationic lipids) adjuvanted PCV composition; the pneumococcal conjugate compositions of the invention, comprising an LNP which includes the cationic lipid CLA provided an equivalent, or increased immunogenic response for the majority of serotypes tested in a pneumococcal conjugate composition. Further provided are methods of making and using the disclosed compositions.

### BRIEF DESCRIPTION OF THE DRAWINGS

FIG. 1: CLA-LNP components: (13Z,16Z)-N,N-dimethyl-3-nonyldocosa-13,16-dien-1-amine (CLA); cholesterol; 1,2-dimyristoyl-rac-glycero-3-methoxypolyethylene glycol-2000 (ePEG2000-DMG); and 1,2-distearoyl-sn-glycero-3-phosphocholine (DSPC).

FIG. 2: The structures of the cationic lipids: 1) CLA or (13Z,16Z)-N,N-dimethyl-3-nonyldocosa-13,16-dien-1-amine; 2) CLX or (6Z,9Z,26Z,29Z)-N,N-dimethylpentatriaconta-6,9,26,29-tetraen-18-amine; and 3) CLY or N,N-dimethyl-1-((1S,2R)-2-octylcyclopropyl)heptadecan-8-amine formulated as LNPs and evaluated with pneumococcal conjugate vaccine formulations.

FIG. 3: Pre-immune (pooled) and post-dose 3 (day 35) IgG titers of mice immunized with the compositions described in Table 4.

FIG. 4A: Ratio of serotype specific IgG titers in infant rhesus macaques following immunization (described in Table 5) with PCV24 formulated with CLA-LNP (liquid or lyophilized) or PCV24 formulated with APA/CLA-LNP compared to PCV24 formulated with APA at post dose 2, day 42.

FIG. 4B: Ratio of serotype specific IgG titers in infant rhesus macaques following immunization (described in Table 5) with PCV24 formulated with CLA-LNP (liquid or lyophilized) or PCV24 formulated with APA/CLA-LNP compared to PCV24 formulated with APA at post dose 3, day 70.

FIG. 4C: Ratio of serotype specific IgG titers in infant rhesus macaques following immunization (described in Table 5) with PCV24 formulated with CLA-LNP (liquid or lyophilized) or PCV24 formulated with APA/CLA-LNP compared to PCV24 formulated with APA at post dose 3, day 84.

### DETAILED DESCRIPTION OF THE INVENTION

It was surprisingly found that a pneumococcal conjugate composition comprising an LNP adjuvant provided a comparable or enhanced immunogenic response compared to a pneumococcal conjugate composition comprising a standard aluminum adjuvant. It was further surprisingly found that an LNP adjuvant containing the cationic lipid: (13Z,16Z)-N,N-dimethyl-3-nonyldocosa-13,16-dien-1-amine (CLA) provided an enhanced immunogenic response in a pneumococcal conjugate composition compared to pneumococcal conjugate compositions comprising LNP adjuvants containing other cationic lipids.

The present invention provides a pneumococcal conjugate composition comprising Streptococcus pneumoniae polysaccharide-protein conjugates and a lipid nanoparticle (LNP).

The present invention further provides a pneumococcal conjugate composition comprising Streptococcus pneumoniae polysaccharide-protein conjugates and an LNP, and a pharmaceutically acceptable carrier.

The present invention provides a pneumococcal conjugate composition comprising Streptococcus pneumoniae polysaccharide-protein conjugates and an LNP comprising a cationic lipid, a neutral lipid, a phospholipid, and a PEG-lipid.

The present invention further provides a pneumococcal conjugate composition comprising Streptococcus pneumoniae polysaccharide-protein conjugates and an LNP, wherein the LNP comprises a cationic lipid selected from: (13Z,16Z)-N,N-dimethyl-3-nonyldocosa-13,16-dien-1-amine (referred to as “CLA” and when incorporated into an LNP referred to as “CLA-LNP”); (6Z,9Z,26Z,29Z)-N,N-dimethylpentatriaconta-6,9,26,29-tetraen-18-amine (referred to as “CLX” and when incorporated into an LNP referred to as “CLX-LNP”); and N,N-dimethyl-1-((1S,2R)-2-octylcyclopropyl)heptadecan-8-amine (referred to as “CLY” and when incorporated into an LNP referred to as “CLY-LNP”).

The present invention further provides a pneumococcal conjugate composition comprising Streptococcus pneumoniae polysaccharide-protein conjugates and an LNP, wherein the LNP (CLA-LNP) comprises the cationic lipid (13Z,16Z)-N,N-dimethyl-3-nonyldocosa-13,16-dien-1-amine (CLA).

The present invention further provides a pneumococcal conjugate vaccine comprising 1) Streptococcus pneumoniae polysaccharide-protein conjugates and 2) an LNP comprising i) (13Z,16Z)-N,N-dimethyl-3-nonyldocosa-13,16-dien-1-amine (CLA); ii) cholesterol; (iii) ePEG2000-DMG; and iv) distearoyl phosphatidyl choline (DSPC).

The present invention further provides a pneumococcal conjugate vaccine comprising 1) Streptococcus pneumoniae polysaccharide-protein conjugates and 2) an LNP comprising i) (13Z,16Z)-N,N-dimethyl-3-nonyldocosa-13,16-dien-1-amine (CLA); ii) cholesterol; (iii) ePEG2000-DMG; and iv) distearoyl phosphatidyl choline (DSPC); wherein the pneumococcal conjugates and the LNP are field-mixed (i.e., formulated separately and mixed together prior to administration to a patient). In such embodiment, the Streptococcus pneumoniae polysaccharide-protein conjugates may be present, prior to field-mixing, as a pneumococcal conjugate composition.

The present invention further provides a pneumococcal conjugate vaccine comprising i) Streptococcus pneumoniae polysaccharide-protein conjugates; ii) (13Z,16Z)-N,N-dimethyl-3-nonyldocosa-13,16-dien-1-amine (CLA); iii) cholesterol; (iv) ePEG2000-DMG; and v) distearoyl phosphatidyl choline (DSPC).

In one embodiment, each of the S. pneumoniae polysaccharide-protein conjugates in the composition comprises a polysaccharide of a particular S. pneumonia serotype, wherein the polysaccharides in the conjugates comprise one or more serotypes selected from any known serotype, including, but not limited to serotypes 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20, 20A, 20B, 22F, 23A, 23B, 23F, 24F, 33F, 35B, 35F, and 38. In another embodiment, the serotypes comprise, consist essentially of, or consist of 4, 6B, 9V, 14, 18C, 19F and 23F. In another embodiment, the serotypes comprise, consist essentially of, or consist of 1, 3, 4, 5, 6A, 6B, 7F, 9V, 14, 18C, 19A, 19F and 23F. In another embodiment, the serotypes comprise, consist essentially of, or consist of 1, 3, 4, 5, 6A, 6B, 7F, 9V, 14, 18C, 19A, 19F, 22F, 23F and 33F. In another embodiment, the serotypes comprise, consist essentially of, or consist of 1, 3, 4, 5, 6A, 6B, 7F, 8, 9V, 10A, 11A, 12F, 14, 15B, 18C, 19A, 19F, 22F, 23F and 33F. In another embodiment, the serotypes comprise, consist essentially of, or consist of 1, 3, 4, 5, 6A, 6B, 7F, 8, 9V, 10A, 11A, 12F, 14, 15A, 15B, 18C, 19A, 19F, 22F, 23B, 23F, 24F, 33F and 35B. In another embodiment, the serotypes comprise, consist essentially of, or consist of 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B. In another embodiment, the serotypes comprise, consist essentially of, or consist of 1, 3, 4, 5, 6A, 6B, 7F, 8, 9V, 10A, 11A, 12F, 14, 15A, 15C, 18C, 19A, 19F, 22F, 23B, 23F, 24F, 33F and 35B. In another embodiment the serotypes comprise, consist essentially of, or consist of 3, 6A, 7F, 8, 9N, 10A, IA, 12F, 15A, 15B, 16F, 17F, 19A, 20A, 22F, 23A, 23B, 24F, 31, 33F and 35B. In another embodiment the serotypes comprise, consist essentially of, or consist of 3, 6A, 7F, 8, 9N, 10A, IA, 12F, 15A, de-O-acetylated-15B, 16F, 17F, 19A, 20A, 22F, 23A, 23B, 24F, 31, 33F and 35B. In another embodiment the serotypes comprise, consist essentially of, or consist of 3, 6A, 7F, 8, 9N, 10A, IA, 12F, 15A, 15C, 16F, 17F, 19A, 20A, 22F, 23A, 23B, 24F, 31, 33F and 35B. In another embodiment the serotypes comprise, consist essentially of, or consist of 3, 6A, 7F, 8, 9N, 10A, IA, 12F, 15A, 15B, 16F, 17F, 19A, 20, 22F, 23A, 23B, 24F, 31, 33F and 35B. In another embodiment the serotypes comprise, consist essentially of, or consist of 3, 6A, 7F, 8, 9N, 10A, IA, 12F, 15A, de-O-acetylated-15B, 16F, 17F, 19A, 20, 22F, 23A, 23B, 24F, 31, 33F and 35B. In another embodiment the serotypes comprise, consist essentially of, or consist of 3, 6A, 7F, 8, 9N, 10A, IA, 12F, 15A, 15C, 16F, 17F, 19A, 20, 22F, 23A, 23B, 24F, 31, 33F and 35B. In another embodiment the serotypes comprise, consist essentially of, or consist of 3, 6A, 7F, 8, 9N, 10A, IA, 12F, 15A, 15B, 16F, 17F, 19A, 20B, 22F, 23A, 23B, 24F, 31, 33F and 35B. In another embodiment the serotypes comprise, consist essentially of, or consist of 3, 6A, 7F, 8, 9N, 10A, 11A, 12F, 15A, de-O-acetylated-15B, 16F, 17F, 19A, 20B, 22F, 23A, 23B, 24F, 31, 33F and 35B. In another embodiment the serotypes comprise, consist essentially of, or consist of 3, 6A, 7F, 8, 9N, 10A, IA, 12F, 15A, 15C, 16F, 17F, 19A, 20B, 22F, 23A, 23B, 24F, 31, 33F and 35B.

In one embodiment, the polysaccharide-protein conjugates comprise polysaccharides that are selected from a group of pneumococcal serotypes which consist of serotypes: 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A and 20B), 22F, 23A, 23B, 23F, 24F, 33F, 35B, 35F, or 38. In another embodiment, the group of serotypes consists of 4, 6B, 9V, 14, 18C, 19F and 23F. In another embodiment, the group of serotypes consists of 1, 3, 4, 5, 6A, 6B, 7F, 9V, 14, 18C, 19A, 19F and 23F. In another embodiment, the group of serotypes consists of 1, 3, 4, 5, 6A, 6B, 7F, 9V, 14, 18C, 19A, 19F, 22F, 23F and 33F. In another embodiment, the group of serotypes consists of 1, 3, 4, 5, 6A, 6B, 7F, 8, 9V, 10A, 11A, 12F, 14, 15B, 18C, 19A, 19F, 22F, 23F and 33F. In another embodiment, the group of serotypes consists of 1, 3, 4, 5, 6A, 6B, 7F, 8, 9V, 10A, 11A, 12F, 14, 15A, 15B, 18C, 19A, 19F, 22F, 23B, 23F, 24F, 33F and 35B. In another embodiment, the group of serotypes consists of 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B. In another embodiment, the group of serotypes consists of 1, 3, 4, 5, 6A, 6B, 7F, 8, 9V, 10A, 11A, 12F, 14, 15A, 15C, 18C, 19A, 19F, 22F, 23B, 23F, 24F, 33F and 35B. In another embodiment the group of serotypes consists of 3, 6A, 7F, 8, 9N, 10A, IA, 12F, 15A, 15B, 16F, 17F, 19A, 20A, 22F, 23A, 23B, 24F, 31, 33F and 35B. In another embodiment the group of serotypes consists of 3, 6A, 7F, 8, 9N, 10A, IA, 12F, 15A, de-O-acetylated-15B, 16F, 17F, 19A, 20A, 22F, 23A, 23B, 24F, 31, 33F and 35B. In another embodiment the group of serotypes consists of 3, 6A, 7F, 8, 9N, 10A, IA, 12F, 15A, 15C, 16F, 17F, 19A, 20A, 22F, 23A, 23B, 24F, 31, 33F and 35B. In another embodiment the group of serotypes consists of 3, 6A, 7F, 8, 9N, 10A, IA, 12F, 15A, 15B, 16F, 17F, 19A, 20, 22F, 23A, 23B, 24F, 31, 33F and 35B. In another embodiment the group of serotypes consists of 3, 6A, 7F, 8, 9N, 10A, 11A, 12F, 15A, de-O-acetylated-15B, 16F, 17F, 19A, 20, 22F, 23A, 23B, 24F, 31, 33F and 35B. In another embodiment the group of serotypes consists of 3, 6A, 7F, 8, 9N, 10A, 11A, 12F, 15A, 15C, 16F, 17F, 19A, 20, 22F, 23A, 23B, 24F, 31, 33F and 35B. In another embodiment the group of serotypes consists of 3, 6A, 7F, 8, 9N, 10A, IA, 12F, 15A, 15B, 16F, 17F, 19A, 20B, 22F, 23A, 23B, 24F, 31, 33F and 35B. In another embodiment the group of serotypes consists of 3, 6A, 7F, 8, 9N, 10A, IA, 12F, 15A, de-O-acetylated-15B, 16F, 17F, 19A, 20B, 22F, 23A, 23B, 24F, 31, 33F and 35B. In another embodiment the group of serotypes consists of 3, 6A, 7F, 8, 9N, 10A, 11A, 12F, 15A, 15C, 16F, 17F, 19A, 20B, 22F, 23A, 23B, 24F, 31, 33F and 35B.

In an embodiment, the present invention provides a pneumococcal conjugate composition comprising 7 distinct Streptococcus pneumoniae polysaccharide-protein conjugates, wherein the S. pneumoniae polysaccharides consist of serotypes 4, 6B, 9V, 14, 18C, 19F and 23F.

In an embodiment, the present invention provides a pneumococcal conjugate composition comprising 13 distinct Streptococcus pneumoniae polysaccharide-protein conjugates, wherein the S. pneumoniae polysaccharides consist of serotypes 1, 3, 4, 5, 6A, 6B, 7F, 9V, 14, 18C, 19A, 19F and 23F.

In an embodiment, the present invention provides a pneumococcal conjugate composition comprising 15 distinct Streptococcus pneumoniae polysaccharide-protein conjugates, wherein the S. pneumoniae polysaccharides consist of serotypes 1, 3, 4, 5, 6A, 6B, 7F, 9V, 14, 18C, 19A, 19F, 22F, 23F and 33F.

In an embodiment, the present invention provides a pneumococcal conjugate composition comprising 20 distinct Streptococcus pneumoniae polysaccharide-protein conjugates, wherein the S. pneumoniae polysaccharides consist of serotypes 1, 3, 4, 5, 6A, 6B, 7F, 8, 9V, 10A, 11A, 12F, 14, 15B, 18C, 19A, 19F, 22F, 23F and 33F.

In an embodiment, the present invention provides a pneumococcal conjugate composition comprising 24 distinct Streptococcus pneumoniae polysaccharide-protein conjugates, wherein the S. pneumoniae polysaccharides consist of serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B.

In an embodiment, the present invention provides a pneumococcal conjugate composition comprising 24 distinct Streptococcus pneumoniae polysaccharide-protein conjugates, wherein the S. pneumoniae polysaccharides consist of serotypes 1, 3, 4, 5, 6A, 6B, 7F, 8, 9V, 10A, IA, 12F, 14, 15A, de-O-acetylated-15B, 18C, 19A, 19F, 22F, 23B, 23F, 24F, 33F and 35B.

In an embodiment, the present invention provides a pneumococcal conjugate composition comprising 24 distinct Streptococcus pneumoniae polysaccharide-protein conjugates, wherein the S. pneumoniae polysaccharides consist of serotypes 1, 3, 4, 5, 6A, 6B, 7F, 8, 9V, 10A, IA, 12F, 14, 15A, 15C, 18C, 19A, 19F, 22F, 23B, 23F, 24F, 33F and 35B.

In an embodiment, the present invention provides a pneumococcal conjugate composition comprising 21 distinct Streptococcus pneumoniae polysaccharide-protein conjugates, wherein the S. pneumoniae polysaccharides consist of serotypes 3, 6A, 7F, 8, 9N, 10A, IA, 12F, 15A, 15B, 16F, 17F, 19A, 20A, 22F, 23A, 23B, 24F, 31, 33F and 35B.

In an embodiment, the present invention provides a pneumococcal conjugate composition comprising 21 distinct Streptococcus pneumoniae polysaccharide-protein conjugates, wherein the S. pneumoniae polysaccharides consist of serotypes 3, 6A, 7F, 8, 9N, 10A, IA, 12F, 15A, de-O-acetylated-15B, 16F, 17F, 19A, 20A, 22F, 23A, 23B, 24F, 31, 33F and 35B.

In an embodiment, the present invention provides a pneumococcal conjugate composition comprising 21 distinct Streptococcus pneumoniae polysaccharide-protein conjugates, wherein the S. pneumoniae polysaccharides consist of serotypes 3, 6A, 7F, 8, 9N, 10A, IA, 12F, 15A, 15C, 16F, 17F, 19A, 20A, 22F, 23A, 23B, 24F, 31, 33F and 35B.

In an embodiment, the present invention provides a pneumococcal conjugate composition comprising 21 distinct Streptococcus pneumoniae polysaccharide-protein conjugates, wherein the S. pneumoniae polysaccharides consist of serotypes 3, 6A, 7F, 8, 9N, 10A, 11A, 12F, 15A, 15B, 16F, 17F, 19A, 20, 22F, 23A, 23B, 24F, 31, 33F and 35B.

In an embodiment, the present invention provides a pneumococcal conjugate composition comprising 21 distinct Streptococcus pneumoniae polysaccharide-protein conjugates, wherein the S. pneumoniae polysaccharides consist of serotypes 3, 6A, 7F, 8, 9N, 10A, IA, 12F, 15A, de-O-acetylated-15B, 16F, 17F, 19A, 20, 22F, 23A, 23B, 24F, 31, 33F and 35B.

In an embodiment, the present invention provides a pneumococcal conjugate composition comprising 21 distinct Streptococcus pneumoniae polysaccharide-protein conjugates, wherein the S. pneumoniae polysaccharides consist of serotypes 3, 6A, 7F, 8, 9N, 10A, IA, 12F, 15A, 15C, 16F, 17F, 19A, 20, 22F, 23A, 23B, 24F, 31, 33F and 35B.

In an embodiment, the present invention provides a pneumococcal conjugate composition comprising 21 distinct Streptococcus pneumoniae polysaccharide-protein conjugates, wherein the S. pneumoniae polysaccharides consist of serotypes 3, 6A, 7F, 8, 9N, 10A, IA, 12F, 15A, 15B, 16F, 17F, 19A, 20B, 22F, 23A, 23B, 24F, 31, 33F and 35B.

In an embodiment, the present invention provides a pneumococcal conjugate composition comprising 21 distinct Streptococcus pneumoniae polysaccharide-protein conjugates, wherein the S. pneumoniae polysaccharides consist of serotypes 3, 6A, 7F, 8, 9N, 10A, IA, 12F, 15A, de-O-acetylated-15B, 16F, 17F, 19A, 20B, 22F, 23A, 23B, 24F, 31, 33F and 35B.

In an embodiment, the present invention provides a pneumococcal conjugate composition comprising 21 distinct Streptococcus pneumoniae polysaccharide-protein conjugates, wherein the S. pneumoniae polysaccharides consist of serotypes 3, 6A, 7F, 8, 9N, 10A, IA, 12F, 15A, 15C, 16F, 17F, 19A, 20B, 22F, 23A, 23B, 24F, 31, 33F and 35B. In an embodiment of the compositions above, the compositions do not comprise polysaccharide-protein conjugates containing polysaccharides of any other S. pneumoniae serotype.

In an embodiment, the protein is a carrier protein selected from OMPC, PhtD, pLys, DT (Diphtheria toxoid), TT (tetanus toxoid), fragment C of TT, pertussis toxoid, cholera toxoid and CRM197. In another embodiment, the carrier protein is CRM197.

In an embodiment, the molar ratio of the LNP components (cationic lipid: cholesterol: ePEG2000-DMG: and DSPC) is 58:30:10:2, respectively.

The present invention also provides a method of making a pneumococcal conjugate composition comprising Streptococcus pneumoniae polysaccharide-protein conjugates and an LNP.

The present invention also provides methods of treatment or prevention of pneumococcal diseases with a pneumococcal conjugate composition of the instant invention.

The present invention also provides a use of the pneumococcal conjugate composition of the instant invention for the treatment or prevention of pneumococcal diseases.

### Definitions

As used throughout the specification and in the appended claims, the singular forms “a,” “an,” and “the” include the plural reference unless the context clearly dictates otherwise.

As used throughout the specification and appended claims, the following abbreviations and definitions apply:
APA aluminum phosphate adjuvant
CLA ((13Z,16Z)-N,N-dimethyl-3-nonyldocosa-13,16-dien-1-amine)
DMSO dimethylsulfoxide
DSPC 1,2-distearoyl-sn-glycero-3-phosphocholine
ePEG2000-DMG α-[8′-(1,2-Dimyristoyl-3-propanoxy)-carboxamide-3′, 6′-Dioxaoctanyl]carbamoyl-ω-methyl-poly(ethylene glycol)-2000
HPSEC high performance size exclusion chromatography
LNP lipid nanoparticle
NMWCO nominal molecular weight cut off
PCV pneumococcal conjugate vaccine
PD1 post dose 1
PD2 post dose 2
PD3 post dose 3
PnPs pneumococcal polysaccharide
Ps polysaccharide
PS-20 polysorbate-20
w/v weight per volume

As used herein, the term “about,” when used herein in reference to a value, refers to a value that is the same as or, in context, is similar to the referenced value. In general, those skilled in the art, familiar with the context, will appreciate the absolute amount and/or relative degree of difference encompassed by “about” in that context. For example, in some embodiments, the term “about” can encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

As used herein, the term “alkenyl” refers to a straight chain, cyclic or branched unsaturated aliphatic hydrocarbon having the specified number of carbon atoms. In one embodiment, an alkenyl group contains from 8 to 24 carbon atoms (C₈-C₂₄ alkenyl). In one embodiment, an alkenyl group is linear. In another embodiment, an alkenyl group is branched. In another embodiment the alkenyl group is unsubstituted.

As used herein, the term “alkyl” refers to a straight chain, cyclic or branched saturated aliphatic hydrocarbon having the specified number of carbon atoms. In one embodiment, an alkyl group contains from 8 to 24 carbon atoms (C₈-C₂₄ alkyl). In one embodiment, an alkyl group is linear. In another embodiment, an alkyl group is branched. In another embodiment the alkyl group is unsubstituted.

An “adjuvant,” as defined herein, is a substance that serves to enhance (e.g., increase, accelerate, prolong or modulate) the immunogenicity of a composition of the invention. As disclosed herein, an LNP is to be used as the adjuvant in accordance with the instant invention. An adjuvant may enhance an immune response to an antigen that is weakly immunogenic when administered alone, e.g., inducing no or weak antibody titers or cell-mediated immune response, increase antibody titers to the antigen, and/or lowers the dose of the antigen effective to achieve an immune response in the individual. As used herein, an “adjuvanted composition” is a composition that comprises an adjuvant.

As used herein, the term “administration” refers to the act of providing an active agent, composition, or formulation to a subject. Exemplary routes of administration to the human body can be through the eyes (ophthalmic), mouth (oral), skin (transdermal), nose (nasal), lungs (inhalant), rectal, vaginal, oral mucosa (buccal), ear, by injection (e.g., intravenously (IV), subcutaneously, intratumorally, intraperitoneally, intramuscular (IM), intradermal (ID) etc.) and the like.

The term “antigen” refers to any antigen that can generate one or more immune responses. The antigen may be a protein (including recombinant proteins), polypeptide, or peptide (including synthetic peptides). In certain embodiments, the antigen is a lipid or a carbohydrate (polysaccharide). In certain embodiments, the antigen is a protein extract, cell (including tumor cell), or tissue. The antigen may be one that generates a humoral and/or CTL immune response. Antigens of the instant invention are S. pneumoniae polysaccharides.

The term “cationic lipid” refers to a lipid species that carries a net positive charge at a selected pH, such as physiological pH. Those of skill in the art will appreciate that a cationic lipid can include, but are not limited to, those disclosed in US Patent Application Publication Nos. US2008/0085870, US2008/0057080, US2009/0263407, US2009/0285881, US2010/0055168, US2010/0055169, US2010/0063135, US2010/0076055, US2010/0099738, US2010/0104629, US2013/0017239, US2016/0361411, WO2011/022460, WO2012/040184, WO2011/076807, WO2010/021865, WO 2009/132131, WO2010/042877, WO2010/146740, WO2010/105209, U.S. Pat. Nos. 5,208,036, 5,264,618, 5,279,833, 5,283,185, 6,890,557, and 9,669,097.

As used herein, the term “composition” refers to a formulation containing an active pharmaceutical or biological ingredient (for example, a pneumococcal polysaccharide-protein conjugate and an LNP), along with one or more additional components. The term “composition” is used interchangeably with “pharmaceutical composition” and “formulation”. The compositions can be liquid or solid (e.g. lyophilized). Additional components that may be included as appropriate include pharmaceutically acceptable excipients, additives, diluents, buffers, sugars, amino acids, chelating agents, surfactants, polyols, bulking agents, stabilizers, lyo-protectants, solubilizers, emulsifiers, salts, adjuvants, tonicity enhancing agents, delivery vehicles, and anti-microbial preservatives. Compositions are nontoxic to recipients at the dosages and concentrations employed.

The term “comprises” when used with the compositions of the invention refers to the inclusion of any other components, such as adjuvants and excipients, or the addition of one or more polysaccharide-protein conjugates that are not specifically enumerated.

The term “consisting of” when used with the multivalent polysaccharide-protein conjugate mixture refers to a mixture having those particular S. pneumoniae polysaccharide-protein conjugates and no other S. pneumoniae polysaccharide-protein conjugates from a different serotype.

“Consists essentially of” and variations such as “consist essentially of” or “consisting essentially of,” indicate the inclusion of any recited elements or group of elements, and the optional inclusion of other elements, of similar or different nature than the recited elements, which do not materially change the basic or novel properties of the specified dosage regimen, method, or composition.

As used herein, the term “de-O-acetylated-15B” or “de-O-acetyl-15B” or “de-O-Ac-15B” refers to a de-O-acetylated serotype 15B wherein the O-Acetyl content is less than 5% per repeating unit. In another embodiment the O-Acetyl content is less than 1% per repeating unit. In another embodiment the O-Acetyl content is less than 0.5% per repeating unit. In another embodiment the O-Acetyl content is less than 0.1% per repeating unit. Processes for de-O-acetylation are known in the art, for example as described in Rajam et al., Clinical and Vaccine Immunology, 2007, 14(9):1223-1227.

As used herein, the term “dose” means a quantity of an agent, API (active pharmaceutical ingredient), formulation, or composition taken or recommended to be taken at a particular time.

As used herein, the term “immunogenic” or “immunogenicity” refers to the ability of an antigen to provoke an immune response in a subject. The term “immunogenic composition” refers to the ability of an agent, API, formulation, or composition to provoke an immune response in a subject. The pneumococcal conjugate compositions of the instant invention are immunogenic compositions.

Those “in need of treatment” include those previously exposed to or infected with S. pneumoniae, those who were previously vaccinated against S. pneumoniae, as well as those prone to have an infection or any person in which a reduction in the likelihood of infection is desired, e.g., the immunocompromised, the elderly, children, adults, or healthy individuals.

The phrase “indicated for the prevention of pneumococcal disease” means that a vaccine or composition is approved by one or more regulatory authorities, such as the US Food and Drug Administration, for the prophylaxis of one or more diseases caused by any serotype of S. pneumoniae, including, but not limited to: pneumococcal disease generally, pneumococcal pneumonia, pneumococcal meningitis, pneumococcal bacteremia, invasive disease caused by S. pneumoniae, and otitis media caused by S. pneumoniae.

As used herein, the term “lipid” refers to any of a group of organic compounds that are esters of fatty acids and are characterized by being insoluble in water or having low solubility in water but may be soluble in many organic solvents. Lipids can be divided in at least three classes: (1) “simple lipids,” which include, e.g., fats and oils as well as waxes; (2) “compound lipids,” which include, e.g., phospholipids and glycolipids; and (3) “derived lipids,” which include, e.g., steroids.

As used herein, the term “lipid nanoparticle” (or “LNP”) refers to a lipid composition that forms a particle having a length or width measurement (e.g., a maximum length or width measurement) between 10 and 1000 nanometers, and comprises more than one class and/or type of lipid. For example, according to the instant invention, a lipid nanoparticle cannot be solely comprised of cationic lipids. In embodiments, the LNP is used as an adjuvant to increase or enhance the immune response against an antigen of interest when used as a component of a composition and/or vaccine of the instant invention. In embodiments, an LNP is used as an adjuvant and may be used in combination with non-LNP adjuvants.

As used herein, the term “neutral lipid” refers to a lipid species that exists either in an uncharged or neutral zwitterionic form at a selected pH. At physiological pH, such lipids include, for example, diaeylphosphatidylcholine, diacylphosphatidyletbanolamine, ceramide, sphingomyelin, cephalin, cholesterol, cerebrosides and diacylglycerols.

A “patient” (alternatively referred to herein as a “subject”) refers to a mammal capable of being infected with S. pneumoniae. In preferred embodiments, the patient is a human. A patient can be treated prophylactically or therapeutically. Prophylactic treatment provides sufficient protective immunity to reduce the likelihood or severity of a pneumococcal infection or the effects thereof, e.g., pneumococcal pneumonia. Therapeutic treatment can be performed to reduce the severity or prevent recurrence of a S. pneumoniae infection or the clinical effects thereof. Prophylactic treatment can be performed using a pneumococcal conjugate composition or vaccine of the invention, as described herein. The pneumococcal conjugate compositions or vaccines of the invention can be administered to the general population or to those persons at an increased risk of pneumococcal infection, e.g. the elderly, or those who live with or care for the elderly.

“PCV1,” as used herein, refers to a 1-valent pneumococcal conjugate vaccine or composition comprising one S. pneumoniae polysaccharide-protein conjugate, which conjugate comprises capsular polysaccharide from a S. pneumoniae serotype conjugated to a carrier protein. In specific embodiments, the carrier protein is CRM197.

“PCV22,” as used herein, refers to a 22-valent pneumococcal conjugate vaccine or composition comprising twenty-one S. pneumoniae polysaccharide-protein conjugates, each comprising capsular polysaccharide from a S. pneumoniae serotype conjugated to a carrier protein, wherein the serotypes of S. pneumoniae are: 1, 3, 4, 5, 6A, 6B, 7F, 9V, 10A, 12F, 14, 15A, 18C, 19A, 19F, 22F, 23B, 24F, 31, 33F and 35B, and at least one of the following serogroup 15 serotypes: 15B, 15C or de-O-acetylated-15B. In a particular embodiment, the serogroup 15 serotype is serotype 15C or de-O-acetylated-15B. In another embodiment, the serogroup 15 serotype is a de-O-acetylated-15B serotype. In specific embodiments, the carrier protein of one or more of the S. pneumoniae polysaccharide-protein conjugates is CRM197. In further embodiments, the carrier protein of each of the S. pneumoniae polysaccharide-protein conjugates is CRM197.

“PCV24,” as used herein, refers to a 24-valent pneumococcal conjugate vaccine or composition comprising twenty-three S. pneumoniae polysaccharide-protein conjugates, each comprising capsular polysaccharide from a S. pneumoniae serotype conjugated to a carrier protein, wherein the serotypes of S. pneumoniae are: 1, 3, 4, 5, 6A, 6B, 7F, 8, 9V, 10A, IA, 12F, 14, 15A, 18C, 19A, 19F, 22F, 23B, 23F, 24F, 33F and 35B, and one of the following serogroup 15 serotypes: 15B, 15C or de-O-acetylated-15B. In a particular embodiment, the serogroup 15 serotype is serotype 15C or de-O-acetylated-15B. In another embodiment, the serogroup 15 serotype is serotype de-O-acetylated-15B. In specific embodiments, the carrier protein of one or more of the S. pneumoniae polysaccharide-protein conjugates is CRM197. In further embodiments, the carrier protein of each of the S. pneumoniae polysaccharide-protein conjugates is CRM197.

With respect to a carrier, diluent, or excipient of a composition, the term “pharmaceutically acceptable” indicates that a carrier, diluent, or excipient must be compatible with the other ingredients of the composition and not deleterious to the recipient thereof.

As used herein, the terms “pneumococcal conjugate” or “pneumococcal polysaccharide-protein conjugate” refer to an S. pneumoniae polysaccharide-protein conjugate.

As used herein, the term “pneumococcal conjugate vaccine” (or “PCV”) is a pharmaceutical preparation or composition comprising pneumococcal polysaccharide-protein conjugate(s) that provide active immunity to disease or pathological conditions caused by serotype(s) of S. pneumoniae.

The term “therapeutically effective amount” refers to an amount of the composition or vaccine sufficient to produce the desired therapeutic effect in a human or animal, e.g. the amount necessary to elicit an immune response, treat, cure, prevent, or inhibit development and progression of a disease or the symptoms thereof and/or the amount necessary to ameliorate symptoms or cause regression of a disease. One of skill in the art can readily determine a therapeutically effective amount of a given composition or vaccine.

As used herein, the term “valent” refers to the presence of a specified number of polysaccharide-protein conjugates in a composition.

As used herein, the term “vaccine” or “vaccine composition” refers to a biological preparation used to stimulate the production of antibodies and provide immunity against an infectious disease.

### LNPs and LNP Adjuvants

LNPs are known and described in the following publications: U.S. Pat. No. 7,691,405, US2006/0083780, US2006/0240554, US2008/0020058, US2009/0263407, US2009/0285881, WO2009/086558, WO2009/127060, WO2009/132131, WO2010/042877, WO2010/054384, WO2010/054401, WO2010/054405 and WO2010/054406.

LNPs are used herein to boost the immunological response of a pneumococcal conjugate vaccine or composition. Generally, LNPs include one or more cationic lipids, one or more poly(ethylneglycol)-lipids (PEG-lipid), one or more cholesterol molecules, and one or more phospholipids.

LNPs may include any cationic lipid mentioned in the following patent publications: US2008/0085870, US2008/0057080, US2009/0263407, US2009/0285881, US2010/0055168, US2010/0055169, US2010/0063135, US2010/0076055, US2010/0099738, US2010/0104629, US2013/0017239, US2016/0361411, WO2011/022460, WO2012/040184, WO2011/076807, WO2010/021865, WO2009/132131, WO2010/042877, WO2010/146740, WO2010/105209, U.S. Pat. Nos. 5,208,036, 5,264,618, 5,279,833, 5,283,185, 6,890,557, and 9,669,097.

An LNP to be used as an adjuvant in accordance with the instant invention may include a cationic lipid having the following structure, illustrated by Formula 1:

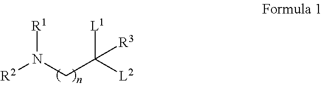

wherein:
R¹ and R² are each methyl;
R³ is H;
n is 1 or 2;
L₁ is selected from C₈-C₂₄ alkyl and C₈-C₂₄ alkenyl; and
L₂ is selected from C₄-C₉ alkyl and C₄-C₉ alkenyl;
or any pharmaceutically acceptable salt or stereoisomer thereof.

A cationic lipid may be an aminoalkyl lipid.

A cationic lipid may be an asymmetric aminoalkyl lipid.

In an embodiment of the instant invention, the LNP comprises a cationic lipid which is (13Z,16Z)-N,N-dimethyl-3-nonyldocosa-13,16-dien-1-amine (CLA); or (6Z,9Z,26Z,29Z)-N,N-dimethylpentatriaconta-6,9,26,29-tetraen-18-amine (CLX); or N,N-dimethyl-1-((1S,2R)-2-octylcyclopropyl)heptadecan-8-amine (CLY).

In another embodiment of the instant invention, the LNP comprises a cationic lipid which is (13Z,16Z)-N,N-dimethyl-3-nonyldocosa-13,16-dien-1-amine (CLA) (See U.S. Pat. No. 9,669,097).

In another embodiment of the instant invention, the cationic lipid is selected from:
DLinDMA;
DLinKC2DMA;
DLin-MC3-DMA;
CLinDMA;
S-Octyl CLinDMA;
(2S)-1-{7-[(3P)-cholest-5-en-3-yloxy]heptyloxy}-3-[(4Z)-dec-4-en-1-yloxy]-N,N-dimethylpropan-2-amine;
(2R)-1-{4-[(3P)-cholest-5-en-3-yloxy]butoxy}-3-[(4Z)-dec-4-en-1-yloxy]-N,N-dimethylpropan-2-amine;
1-[(2R)-1-{4-[(30)-cholest-5-en-3-yloxy]butoxy}-3-(octyloxy)propan-2-yl]guanidine;
1-[(2R)-1-{7-[(3β)-cholest-5-en-3-yloxy]heptyloxy}-N,N-dimethyl-3-[(9Z,12Z)-octadeca-9,12-dien-1-yloxy]propan-2-amine;
1-[(2R)-1-{4-[(30)-cholest-5-en-3-yloxy]butoxy}-N,N-dimethyl-3-[(9Z,12Z)-octadeca-9,12-dien-1-yloxy]propan-2-amine;
(2S)-1-({6-[(3P))-cholest-5-en-3-yloxy]hexyl}oxy)-N,N-dimethyl-3-[(9Z)-octadec-9-en-1-yloxy]propan-2-amine;
(3β)-3-[6-{[(2S)-3-[(9Z)-octadec-9-en-1-yloxyl]-2-(pyrrolidin-1-yl)propyl]oxy}hexyl)oxy]cholest-5-ene;
(2R)-1-{4-[(3P)-cholest-5-en-3-yloxy]butoxy}-3-(octyloxy)propan-2-amine;
(2R)-1-({8-[(3P)-cholest-5-en-3-yloxy]octyl}oxy)-N,N-dimethyl-3-(pentyloxy)propan-2-amine;
(2R)-1-({8-[(3P)-cholest-5-en-3-yloxy]octyl}oxy)-3-(heptyloxy)-N,N-dimethylpropan-2-amine;
(2R)-1-({8-[(3P)-cholest-5-en-3-yloxy]octyl}oxy)-N,N-dimethyl-3-[(2Z)-pent-2-en-1-yloxy]propan-2-amine;
(2S)-1-butoxy-3-({8-[(3P)-cholest-5-en-3-yloxy]octyl}oxy)-N,N-dimethylpropan-2-amine;
(2S-1-({8-[(3P)-cholest-5-en-3-yloxy]octyl}oxy)-3-[2,2, 3,3,4,4,5, 5,6, 6,7, 7, 8, 8,9, 9-hexadecafluorononyl)oxy]-N,N-dimethylpropan-2-amine;
2-amino-2-{[(9Z,12Z)-octadeca-9, 12-dien-1-yloxy]methyl} propane-1,3-diol;
2-amino-3-({9-[(3β,8ξ9ξ,14ξ,17ξ,20ξ)-cholest-5-en-3-yloxy]nonyl}oxy)-2-{[(9Z,12Z)-octadeca-9,12-dien-1-yloxy]methyl}propan-1-ol;
2-amino-3-({6-[(3β,8ξ,9ξ,14ξ,17ξ,20ξ)-cholest-5-en-3-yloxy]nonyl}oxy)-2-{[(9Z)-octadec-9-en-1-yloxy]methyl}propan-1-ol;
(20Z,23Z)-N,N-dimethylnonacosa-20,23-dien-10-amine;
(17Z,20Z)-N,N-dimethylhexacosa-17,20-dien-9-amine;
(16Z,19Z)-N,N-dimethylpentacosa-16,19-dien-8-amine;
(13Z,16Z)-N,N-dimethyldocosa-13,16-dien-5-amine;
(12Z,15Z)-N,N-dimethylhenicosa-12,15-dien-4-amine;
(14Z,17Z)-N,N-dimethyltricosa-14,17-dien-6-amine;
(15Z,18Z)-N,N-dimethyltetracosa-15,18-dien-7-amine;
(18Z,21 Z)-N,N-dimethylheptacosa-18,21-dien-10-amine;
(15Z,18Z)-N,N-dimethyltetracosa-15,18-dien-5-amine;
(14Z,17Z)-N,N-dimethyltricosa-14,17-dien-4-amine;
(19Z,22Z)-N,N-dimethyloctacosa-19,22-dien-9-amine;
(18Z,21Z)-N,N-dimethylheptacosa-18,21-dien-8-amine;
(17Z,20Z)-N,N-dimethylhexacosa-17,20-dien-7-amine;
(16Z,19Z)-N,N-dimethylpentacosa-16,19-dien-6-amine;
(22Z,25Z)-N,N-dimethylhentriaconta-22,25-dien-10-amine;
(21 Z,24Z)-N,N-dimethyltriaconta-21,24-dien-9-amine;
(18Z)-N,N-dimethylheptacos-18-en-10-amine;
(17Z)-N,N-dimethylhexacos-17-en-9-amine;
(19Z,22Z)-N,N-dimethyloctacosa-19,22-dien-7-amine;
N,N-dimethylheptacosan-10-amine;
(20Z,23Z)-N-ethyl-N-methylnonacosa-20,23-dien-10-amine;
1-[(11Z,14Z)-1-nonylicosa-11,14-dien-1-yl]pyrrolidine;
(20Z)-N,N-dimethylheptacos-20-en-10-amine;
(15Z)-N,N-dimethylheptacos-15-en-10-amine;
(14Z)-N,N-dimethylnonacos-14-en-10-amine;
(17Z)-N,N-dimethylnonacos-17-en-10-amine;
(24Z)-N,N-dimethyltritriacont-24-en-10-amine;
(20Z)-N,N-dimethylnonacos-20-en-10-amine;
(22Z)-N,N-dimethylhentriacont-22-en-10-amine;
(16Z)-N,N-dimethylpentacos-16-en-8-amine;
(12Z,15Z)-N,N-dimethyl-2-nonylhenicosa-12,15-dien-1-amine;
(13Z,16Z)-N,N-dimethyl-3-nonyldocosa-13,16-dien-1-amine;
N,N-dimethyl-1-[(1S,2R)-2-octylcyclopropyl]heptadecan-8-amine;
1-[(1 S,2R)-2-hexylcyclopropyl]-N,N-dimethylnonadecan-10-amine;
N,N-dimethyl-1-[(1S,2R)-2-octylcyclopropyl]nonadecan-10-amine;
N,N-dimethyl-21-[(1S,2R)-2-octylcyclopropyl]henicosan-10-amine;
N,N-dimethyl-1-[(1S,2S)-2-{[(1R,2R)-2-pentylcyclopropyl]methyl}cyclopropyl]nonadecan-10-amine;
N,N-dimethyl-1-[(1S,2R)-2-octylcyclopropyl]hexadecan-8-amine;
N,N-dimethyl-1-[(1R,2S)-2-undecylcyclopropyl]tetradecan-5-amine;
N,N-dimethyl-3-{7-[(1 S,2R)-2-octylcyclopropyl]heptyl}dodecan-1-amine;
1-[(1R,2S)-2-heptylcyclopropyl]-N,N-dimethyloctadecan-9-amine;
1-[(1S,2R)-2-decylcyclopropyl]-N,N-dimethylpentadecan-6-amine;
N,N-dimethyl-1-[(1S,2R)-2-octylcyclopropyl]pentadecan-8-amine; and
(11E,20Z,23Z)-N,N-dimethylnonacosa-11,20,23-trien-10-amine;
   or a pharmaceutically acceptable salt thereof, or a stereoisomer of any of the foregoing.

In some embodiments, the LNP includes 30-65 mole % cationic lipid. In some embodiments, the LNP includes 30-55 mole % cationic lipid. In some embodiments, the LNP includes 30-45 mole % cationic lipid. In some embodiments, the LNP includes 55-65 mole % cationic lipid. In some embodiments, the LNP includes 58 mole % cationic lipid.

In some embodiments of the instant invention, the LNP includes 30-65 mole % CLA. In some embodiments of the instant invention, the LNP includes 30-55 mole % CLA. In some embodiments of the instant invention, the LNP includes 30-45 mole % CLA. In some embodiments of the instant invention, the LNP includes 55-65 mole % CLA. In some embodiments, the LNP includes 58 mole % CLA.

In some embodiments of the instant invention, the LNP includes 30-65 mole % CLX. In some embodiments of the instant invention, the LNP includes 30-55 mole % CLX. In some embodiments of the instant invention, the LNP includes 30-45 mole % CLX. In some embodiments of the instant invention, the LNP includes 55-65 mole % CLX. In some embodiments, the LNP includes 58 mole % CLX.

In some embodiments of the instant invention, the LNP includes 30-65 mole % CLY. In some embodiments of the instant invention, the LNP includes 30-55 mole % CLY. In some embodiments of the instant invention, the LNP includes 30-45 mole % CLY. In some embodiments of the instant invention, the LNP includes 55-65 mole % CLY. In some embodiments, the LNP includes 58 mole % CLY.

An LNP may include a neutral lipid selected from: phospholipids, diaeylphosphatidylcholine, diacylphosphatidyletbanolamine, ceramide, sphingomyelin, cephalin, cholesterol, cerebrosides, diacylglycerols, and combinations thereof. In some embodiments, the neutral lipid may include a phospholipid and cholesterol.

A neutral lipid may include a sterol, such as cholesterol. In some embodiments, the neutral lipid includes cholesterol.

In some embodiments of the instant invention, the LNP includes 10-40 mole % cholesterol. In some embodiments of the instant invention, the LNP includes 15-25 mole % cholesterol. In some embodiments of the instant invention, the LNP includes 10-20 mole % cholesterol. In some embodiments of the instant invention, the LNP includes 20-30 mole % cholesterol. In some embodiments of the instant invention, the LNP includes 10-15 mole % cholesterol. In some embodiments of the instant invention, the LNP includes 25-35 mole % cholesterol. In some embodiments, the LNP includes 30 mole % cholesterol.

An LNP may include a phospholipid selected from: phospholipids, aminolipids and sphingolipids. In some embodiments, the LNP may include a phospholipid selected from: phosphatidylcholine, phosphatidylethanolamine, phosphatidylserine, phosphatidylinositol, phosphatidic acid, palmitoyloleryl phosphatidylcholine, lysophosphatidylcholine, lysophosphatidylethanolamine, dipalmitoylphosphatidylcholine, dioleoylphospbatidylcholine, dstearoylphosphatidylcholine or dilinoleoylphosphatidylcholine. In some embodiments, the LNP adjuvant may include a neutral lipid selected from: sphingolipid, glycosphingolipid families, diacylglycerols and S-acyloxyacids. In some embodiments, the LNP may include a neutral lipid selected from: phosphatidylcholine (PC), phosphatidylethanolamine (PE), and phosphatidylglycerol (PG), phosphatidylserine (PS), phosphatidylinositol (PI), phosphatidic acid (phosphatidate) (PA), dipalmitoylphosphatidylcholine, monoacyl-phosphatidylcholine (lyso PC), 1-palmitoyl-2-oleoyl-sn-glycero-3-phosphocholine (POPC), N-acyl-PE, phosphoinositides, and phosphosphingolipids. In some embodiments, the LNP may include a neutral lipid selected from: phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), and phosphatidylserine (DOPS). In some embodiments, the LNP may include a neutral lipid selected from: fatty acids which include C14:0, palmitic acid (C16:0), stearic acid (C18:0), oleic acid (C18:1), linoleic acid (C18:2), linolenic acid (C18:3), arachidonic acid (C20:4), C20:0, C22:0 and lecithin. In some embodiments, the phospholipid may include 1,2-Distearoyl-sn-glycero-3-phosphocholine (DSPC).

In some embodiments of the instant invention, the neutral lipid may include a phospholipid. In some embodiments of the instant invention, the LNP includes 5-30 mole % phospholipid. In some embodiments of the instant invention, the LNP includes 5-15 mole % phospholipid. In some embodiments of the instant invention, the LNP includes 10-20 mole % phospholipid. In some embodiments of the instant invention, the LNP includes 20-30 mole % phospholipid. In some embodiments of the instant invention, the LNP includes 10-15 mole % phospholipid. In some embodiments of the instant invention, the LNP includes 25-30 mole % phospholipid. In some embodiments, the LNP includes 10 mole % phospholipid.

In some embodiments of the instant invention, the neutral lipid is DSPC. In some embodiments of the instant invention, the LNP includes 5-30 mole % DSPC. In some embodiments of the instant invention, the LNP includes 5-15 mole % DSPC. In some embodiments of the instant invention, the LNP includes 10-20 mole % DSPC. In some embodiments of the instant invention, the LNP includes 20-30 mole % DSPC. In some embodiments of the instant invention, the LNP includes 10-15 mole % DSPC. In some embodiments of the instant invention, the LNP includes 25-30 mole % DSPC. In some embodiments of the instant invention, the LNP includes 10 mole % DSPC.

A polymer-lipid conjugate may include a PEG-lipid. In some embodiments the PEG is conjugated to the lipid via a direct linkage (see, e.g., cPEG2000-DMG described below) or is conjugated to the lipid via linker (see, e.g., ePEG2000-DMG). In some embodiments, the PEG-lipid is conjugated to a diacylglycerol (a PEG-DAG). In some embodiments, the PEG is conjugated to DAG as described in, e.g., U.S. Patent Publication Nos. 2003/0077829 and 2005/008689. In one embodiment, the PEG-DAG conjugate is a PEG dimyristylglycerol (c14) conjugate. In some embodiments, the PEG-lipid is PEG-dimyristolglycerol (PEG-DMG).

In certain embodiments, the PEG-lipid is PEG conjugated to dimyristoylglycerol (PEG-DMG), e.g., as described in Abrams et al., 2010, Molecular Therapy 18(1):171, and U.S. Patent Application Publication Nos. US 2006/0240554 and US 2008/0020058.

In certain embodiments, the PEG-lipid comprises a polyethylene glycol having an average molecular weight raining of about 500 daltons to about 10,000 daltons, of about 75 daltons to about 5,000 daltons, of about 1,000 daltons to about 5,000 daltons, of about 1,500 daltons to about 3,000 daltons or of about 2,000 daltons. In certain embodiments, the PEG-lipid comprises PEG1500, PEG2000 or PEG5000.

In some embodiments, the LNP adjuvant may include a PEG-lipid selected from:
1,2-Dimyristoyl-sn-glycerol methoxy-poly(ethylene glycol);
1,2-Dimyristoyl-sn-glycerol methoxy-poly(ethylene glycol)-2000 (cPEG2000-DMG(s)), which has the following structure:

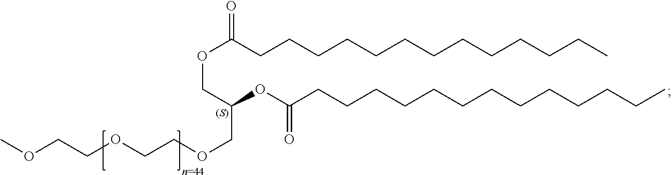

1,2-Dimyristoyl-rac-glycerol methoxy-poly(ethylene glycol);
1,2-Dimyristoyl-rac-glycerol methoxy-poly(ethylene glycol)-2000 (cPEG2000-DMG) which has the following structure:

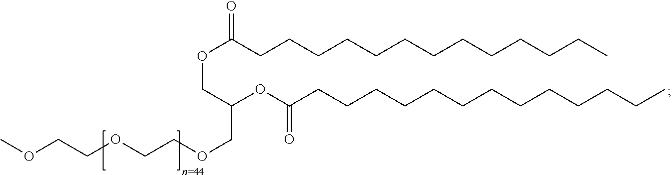

α-[8′-(1,2-Dimyristoyl-3-propanoxy)-carboxamide-3′, 6′-Dioxaoctanyl]carbamoyl-ω-methyl-poly(ethylene glycol);
α-[8′-(1,2-Dimyristoyl-3-propanoxy)-carboxamide-3′, 6′-Dioxaoctanyl]carbamoyl-ω-methyl-poly(ethylene glycol)-2000 (ePEG2000-DMG) which has the following structure:

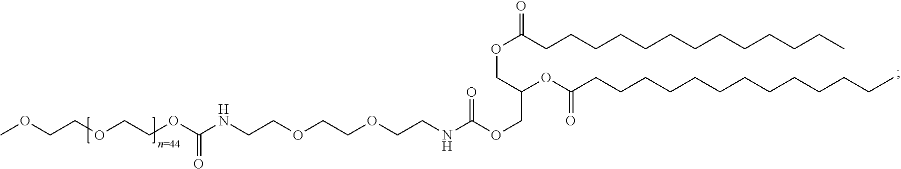

(R)-α-[8′-(1,2-Dimyristoyl-3-propanoxy)-carboxamide-3′, 6′-Dioxaoctanyl]carbamoyl-ω-methyl-poly(ethylene glycol)-2000 which has the following structure:

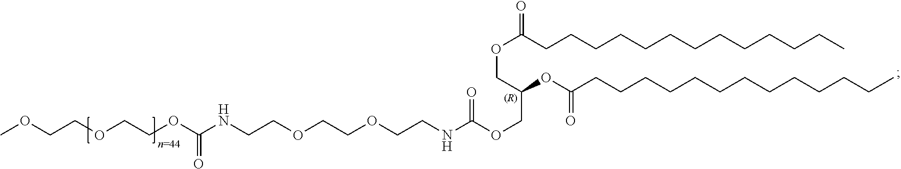

1,2-dimyristoyl-sn-glycero-3-phosphoethanolamine-N-[methoxy(polyethylene glycol);
1,2-dimyristoyl-sn-glycero-3-phosphoethanolamine-N-[methoxy(polyethylene glycol)-2000] which has the following structure:

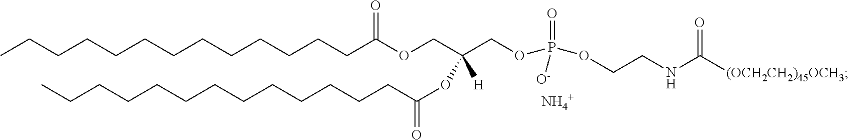

1,2-didecanoyl-rac-glycero-3-methylpolyoxyethylene;
1,2-didodecanoyl-rac-glycero-3-methylpolyoxyethylene; or
1,2-Distearoyl-rac-glycero-3-methylpolyoxyethylene.

In some embodiments of the instant invention, the LNP includes 0.05-15 mole % polymer-lipid conjugate. In some embodiments of the instant invention, the LNP includes 1-4 mole % polymer-lipid conjugate. In some embodiments of the instant invention, the LNP includes 0.5-2 mole % polymer-lipid conjugate. In some embodiments of the instant invention, the LNP includes 1-3 mole % polymer-lipid conjugate. In some embodiments of the instant invention, the LNP includes 1-2.5 mole % polymer-lipid conjugate. In some embodiments, the LNP includes 2 mole % polymer-lipid conjugate. (In each case, mole % is expressed as total mole % of polymer-lipid conjugate in the LNP.)

In some embodiments of the instant invention, the LNP includes 0.05-15 mole % PEG-DMG. In some embodiments of the instant invention, the LNP includes 1-4 mole % PEG-DMG. In some embodiments of the instant invention, the LNP includes 0.5-2 mole % PEG-DMG. In some embodiments of the instant invention, the LNP includes 1-4 mole % PEG-DMG. In some embodiments of the instant invention, the LNP includes 1-3 mole % PEG-DMG. In some embodiments of the instant invention, the LNP includes 1-2.5 mole % PEG-DMG. In some embodiments of the instant invention, the LNP includes 2 mole % PEG-DMG.

In some embodiments of the instant invention, the LNP includes 0.05-15 mole % ePEG2000-DMG. In some embodiments of the instant invention, the LNP includes 1-4 mole % ePEG2000-DMG. In some embodiments of the instant invention, the LNP includes 0.5-2 mole % ePEG2000-DMG. In some embodiments of the instant invention, the LNP includes 1-4 mole % ePEG2000-DMG. In some embodiments of the instant invention, the LNP includes 1-3 mole % ePEG2000-DMG. In some embodiments of the instant invention, the LNP includes 1-2.5 mole % ePEG2000-DMG. In some embodiments of the instant invention, the LNP includes 2 mole % ePEG2000-DMG.

In some embodiments of the instant invention, the LNP includes 30-65 mole % cationic lipid, 10-30 mole % cholesterol, 5-30 mole % phospholipid, and 0.5-4 mole % PEG-lipid. In some embodiments of the instant invention, the LNP includes 55-65 mole % cationic lipid, 25-35 mole % cholesterol, 5-15 mole % phospholipid, and 1-2.5 mole % PEG-lipid. In some embodiments of the instant invention, the LNP includes 40-50 mole % cationic lipid, 15-20 mole % cholesterol, 18-20 mole % phospholipid, and 1.5-2.5 mole % PEG-lipid. In some embodiments of the instant invention, the LNP includes 56-59 mole % cationic lipid, 15-20 mole % cholesterol, 18-20 mole % phospholipid, and 0.5-1.5 mole % PEG-lipid. In some embodiments, the LNP includes 56-59 mole % cationic lipid, 28-32 mole % cholesterol, 8-12 mole % phospholipid, and 1-3 mole % PEG-lipid. In some embodiments, the LNP includes 58 mole % cationic lipid, 30 mole % cholesterol, 10 mole % phospholipid, and 2 mole % PEG-lipid.

In some embodiments of the instant invention, the LNP includes 30-65 mole % CLA, 10-30 mole % cholesterol, 5-30 mole % DSPC, and 0.5-15 mole % ePEG2000-DMG. In some embodiments of the instant invention, the LNP includes 55-65 mole % CLA, 25-35 mole % cholesterol, 5-15 mole % DSPC, and 1-2.5 mole % ePEG2000-DMG. In some embodiments of the instant invention, the LNP includes 40-50 mole % CLA, 15-20 mole % cholesterol, 18-20 mole % DSCP, and 1.5-2.5 mole % ePEG2000-DMG. In some embodiments of the instant invention, the LNP includes 56-59 mole % CLA, 15-20 mole % cholesterol, 18-20 mole % DSPC, and 0.5-1.5 mole % ePEG2000-DMG. In some embodiments, the LNP includes 56-59 mole % CLA, 28-32 mole % cholesterol, 8-12 mole % DSPC, and 1-3 mole % ePEG2000-DMG. In some embodiments, the LNP includes 58 mole % CLA, 30 mole % cholesterol, 10 mole % DSCP, and 2 mole % ePEG2000-DMG.

In some embodiments of the instant invention, the LNP includes 30-65 mole % CLX, 10-30 mole % cholesterol, 5-30 mole % DSPC, and 0.5-15 mole % ePEG2000-DMG. In some embodiments of the instant invention, the LNP includes 55-65 mole % CLX, 25-35 mole % cholesterol, 5-15 mole % DSPC, and 1-2.5 mole % ePEG2000-DMG. In some embodiments of the instant invention, the LNP includes 40-50 mole % CLX, 15-20 mole % cholesterol, 18-20 mole % DSCP, and 1.5-2.5 mole % ePEG2000-DMG. In some embodiments of the instant invention, the LNP includes 56-59 mole % CLX, 15-20 mole % cholesterol, 18-20 mole % DSPC, and 0.5-1.5 mole % ePEG2000-DMG. In some embodiments, the LNP includes 56-59 mole % CLX, 28-32 mole % cholesterol, 8-12 mole % DSPC, and 1-3 mole % ePEG2000-DMG. In some embodiments, the LNP includes 58 mole % CLX, 30 mole % cholesterol, 10 mole % DSCP, and 2 mole % ePEG2000-DMG.

In some embodiments of the instant invention, the LNP includes 30-65 mole % CLY, 10-30 mole % cholesterol, 5-30 mole % DSPC, and 0.5-15 mole % ePEG2000-DMG. In some embodiments of the instant invention, the LNP includes 55-65 mole % CLY, 25-35 mole % cholesterol, 5-15 mole % DSPC, and 1-2.5 mole % ePEG2000-DMG. In some embodiments of the instant invention, the LNP includes 40-50 mole % CLY, 15-20 mole % cholesterol, 18-20 mole % DSCP, and 1.5-2.5 mole % ePEG2000-DMG. In some embodiments of the instant invention, the LNP includes 56-59 mole % CLY, 15-20 mole % cholesterol, 18-20 mole % DSPC, and 0.5-1.5 mole % ePEG2000-DMG. In some embodiments, the LNP includes 56-59 mole % CLY, 28-32 mole % cholesterol, 8-12 mole % DSPC, and 1-3 mole % ePEG2000-DMG.

In some embodiments, the LNP includes 58 mole % CLY, 30 mole % cholesterol, 10 mole % DSCP, and 2 mole % ePEG2000-DMG.

In some embodiments, the LNP consists of 1) a cationic lipid; 2) a sterol; 3) a phospholipid; and 4) a PEG-lipid.

In a further embodiment, the cationic lipid is selected from CLA, CLX and CLY.

In a further embodiment, the sterol is selected from cholesterol, stigmasterol and stigmastanol.

In a further embodiment, the phospholipid is selected from phosphatidylserine, 1,2-Distearoyl-sn-glycero-3-phosphocholine (DSPC), 1,2-dipalmitoleoyl-sn-glycero-3-phosphocholine, 1,2-dimyristoyl-sn-glycero-3-phosphocholine (DMPC), dilauroylphosphatidylcholine (DLPC), 1,2-dieicosenoyl-sn-glycero-3-phosphocholine, and 1,2-dioleoyl-sft-glycero-3-phosphocholine (DOPC).

In a further embodiment, PEG-lipid selected from 1,2-Dimyristoyl-sn-glycerol methoxy-poly(ethylene glycol), 1,2-Dimyristoyl-sn-glycerol methoxy-poly(ethylene glycol)-2000 (cPEG2000-DMG(s)), 1,2-Dimyristoyl-rac-glycerol methoxy-poly(ethylene glycol), 1,2-Dimyristoyl-rac-glycerol methoxy-poly(ethylene glycol)-2000 (cPEG2000-DMG), α-[8′-(1,2-Dimyristoyl-3-propanoxy)-carboxamide-3′, 6′-Dioxaoctanyl] carbamoyl-ω-methyl-poly(ethylene glycol), α-[8′-(1,2-Dimyristoyl-3-propanoxy)-carboxamide-3′, 6′-Dioxaoctanyl]carbamoyl-ω-methyl-poly(ethylene glycol)-2000 (ePEG2000-DMG), (R)-α-[8′-(1,2-Dimyristoyl-3-propanoxy)-carboxamide-3′, 6′-Dioxaoctanyl] carbamoyl-ω-methyl-poly(ethylene glycol)-2000, 1,2-dimyristoyl-sn-glycero-3-phosphoethanolamine-N-[methoxy(polyethylene glycol), 1,2-dimyristoyl-sn-glycero-3-phosphoethanolamine-N-[methoxy(polyethylene glycol)-2000], 1,2-didecanoyl-rac-glycero-3-methylpolyoxyethylene, 1,2-didodecanoyl-rac-glycero-3-methylpolyoxyethylene, and 1,2-Distearoyl-rac-glycero-3-methylpolyoxyethylene.

### General Methods of Making LNP Adjuvants

LNPs may be formed, for example, by a rapid precipitation process that entails micro-mixing the lipid components dissolved in a lower alkanol solution (e.g. ethanol) with an aqueous solution using a confined volume mixing apparatus such as a confined volume T-mixer, a multi-inlet vortex mixer, microfluidics mixer devices, or other. The lipid solution may include one or more cationic lipids, one or more neutral lipid (e.g., phospholipids, DSPC, cholesterol), one or more polymer-lipid conjugate (e.g. ePEG2000-DMG or cPEG2000-DMG) at specific molar ratios in ethanol.

In some embodiments, the aqueous and organic solutions are optionally heated to a temperature in the range of 25° C. to 45° C., preferably 30° C. to 40° C., and then mixed in a confined volume mixer to form the LNP. When a confined volume T-mixer is used, the T-mixer may have an internal diameter range from 0.25 to 10 mm. In some embodiments, the alcohol and aqueous solutions may be delivered to the inlet of the T-mixer using programmable syringe pumps, and with a total flow rate from 0.01 L/minute to 600 L/minute. In some embodiments, the aqueous and alcohol solutions may be combined in the confined-volume mixer with a ratio in the range of 1:1 to 4:1 vol:vol. In some embodiments, the aqueous and alcohol solutions may be combined at a ratio in the range of 1.1:1 to 4:1, 1.2:1 to 4:1, 1.25:1 to 4:1, 1.3:1 to 4:1, 1.5:1 to 4:1, 1.6:1 to 4:1, 1.7:1 to 4:1, 1.8:1 to 4:1, 1.9:1 to 4:1, 2.0:1 to 4:1, 2.5:1 to 4:1, 3.0:1 to 4:1, and 3.5:1 to 4:1.

In some embodiments, the combination of ethanol volume fraction, solution flow rates, lipid(s) concentrations, mixer configuration and internal diameter, and mixer tubing internal diameter utilized at this mixing stage may provide LNPs having a particle size of the between 30 and 300 nm. The resulting LNP suspension may be diluted into higher pH buffers in the range of 6-8.

In some embodiments, the LNPs may also be concentrated and filtered via an ultrafiltration process to remove the alcohol. In some embodiments, the high pH buffer may also be removed and exchanged for a final buffer solution. In some embodiments, the final buffer solution may be selected from a phosphate buffered saline or any buffer system suitable for cryopreservation (for example, buffers containing sucrose, trehalose or combinations thereof). Following filtration, the vialed LNP product may be stored under suitable storage conditions (such as, 2° C. to 8° C., or −80° C. to −20° C. if frozen) or may be lyophilized.

In an embodiment of the instant invention the process of preparing an LNP consists of 4 primary steps: 1) solution preparation of the lipid mixture and an aqueous buffer; 2) LNP formation by means of split stream mixing; 3) ultra-filtration; and 4) filtration.

The lipid components are dissolved in ethanol before being sterile filtered to form a lipid mixture. Several aqueous buffers are also prepared. The lipid mixture and buffer streams are then combined using a T-tube or Y mixer and then, immediately after exit, are diluted and mixed with an aqueous buffer to form an LNP intermediate. The LNP intermediate is then subjected to ultra-filtration to both concentrate the material as well as diafilter the material against a suitable buffer to remove residual ethanol. After the diafiltration, there is a final concentration step performed in order to achieve final target concentration. The LNP bulk is then filtered with a sterilizing filter and stored frozen at −70° C.

### Pneumococcal Conjugate Vaccine Compositions

Pneumococcal conjugate vaccines or compositions have been previously disclosed. See WO2011/100151, WO2019/139692 and WO2020/131763.

Example bacterial capsular polysaccharides from S. pneumoniae are serotypes: 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A and 20B), 22F, 23A, 23B, 23F, 24F, 33F, 35B, 35F, or 38.

### General Method for Making Capsular Polysaccharides

Methods of making pneumococcal conjugate vaccines or compositions have been previously disclosed. See WO2011/100151, WO2019/139692 and WO2020/131763.

Bacterial capsular polysaccharides, particularly those that have been used as antigens, are suitable for use in the invention and can readily be identified by methods for identifying immunogenic and/or antigenic polysaccharides. Example bacterial capsular polysaccharides from S. pneumoniae are serotypes: 1, 2, 3, 4, 5, 6A, 6B, 6C, 7C, 7F, 8, 9N, 9V, 10A, 11A, 12F, 14,15A, 15B, 15C, 16F, 17F, 18C, 19A, 19F, 20 (20A and 20B), 22F, 23A, 23B, 23F, 24F, 33F, 35B, 35F, or 38.

Polysaccharides can be purified by known techniques. The invention is not limited to polysaccharides purified from natural sources, however, and the polysaccharides may be obtained by other methods, such as total or partial synthesis. Capsular polysaccharides from S. pneumoniae can be prepared by standard techniques known to those skilled in the art. For example, polysaccharides can be isolated from bacteria and may be sized to some degree by known methods (see, e.g., European Patent Nos. EP497524 and EP497525); and preferably by microfluidization accomplished using a homogenizer or by chemical hydrolysis. S. pneumoniae strains corresponding to each polysaccharide serotype may be grown in a soy-based medium. The individual polysaccharides may then be purified through standard steps including centrifugation, precipitation, and ultrafiltration. See, e.g., U.S. Patent Application Publication No. 2008/0286838 and U.S. Pat. No. 5,847,112. Polysaccharides can be sized in order to reduce viscosity and/or to improve filterability and the lot-to-lot consistency of subsequent conjugated products.

Purified polysaccharides can be chemically activated to introduce functionalities capable of reacting with a carrier protein using standard techniques. The chemical activation of polysaccharides and subsequent conjugation to a carrier protein are achieved by means described in U.S. Pat. Nos. 4,365,170, 4,673,574 and 4,902,506. Briefly, the pneumococcal polysaccharide is reacted with a periodate-based oxidizing agent such as sodium periodate, potassium periodate, or periodic acid resulting in oxidative cleavage of vicinal hydroxyl groups to generate reactive aldehyde groups. Suitable molar equivalents of periodate (e.g., sodium periodate, sodium metaperiodate and the like) include 0.05 to 0.5 molar equivalents (molar ratio of periodate to polysaccharide repeat unit) or 0.1 to 0.5 molar equivalents. The periodate reaction can be varied from 30 minutes to 24 hours depending on the diol conformation (e.g., acyclic diols, cis diols, trans diols), which controls accessibility of the reactive hydroxyl groups to the sodium periodate.

The term “periodate” includes both periodate and periodic acid; the term also includes both metaperiodate (IO⁴⁻) and orthoperiodate (IO⁶⁻) and includes the various salts of periodate (e.g., sodium periodate and potassium periodate). Capsular polysaccharide may be oxidized in the presence of metaperiodate, or in the presence of sodium periodate (NaIO₄). Further, capsular polysaccharide may be oxidized in the presence of orthoperiodate, or in the presence of periodic acid.

Purified polysaccharides can also be connected to a linker. Once activated or connected to a linker, each capsular polysaccharide may be separately conjugated to a carrier protein to form a glycoconjugate. The polysaccharide conjugates may be prepared by known coupling techniques.

Polysaccharide can be coupled to a linker to form a polysaccharide-linker intermediate in which the free terminus of the linker is an ester group. The linker is therefore one in which at least one terminus is an ester group. The other terminus is selected so that it can react with the polysaccharide to form the polysaccharide-linker intermediate.

Polysaccharide can be coupled to a linker using a primary amine group in the polysaccharide. In this case, the linker typically has an ester group at both termini. This allows the coupling to take place by reacting one of the ester groups with the primary amine group in the polysaccharide by nucleophilic acyl substitution. The reaction results in a polysaccharide-linker intermediate in which the polysaccharide is coupled to the linker via an amide linkage. The linker is therefore a bifunctional linker that provides a first ester group for reacting with the primary amine group in the polysaccharide and a second ester group for reacting with the primary amine group in the carrier molecule. A typical linker is adipic acid N-hydroxysuccinimide diester (SIDEA).

The coupling can also take place indirectly, i.e. with an additional linker that is used to derivatize the polysaccharide prior to coupling to the linker.

Polysaccharide can be coupled to the additional linker using a carbonyl group at the reducing terminus of the polysaccharide. This coupling comprises two steps: (a1) reacting the carbonyl group with the additional linker; and (a2) reacting the free terminus of the additional linker with the linker. In these embodiments, the additional linker typically has a primary amine group at both termini, thereby allowing step (a1) to take place by reacting one of the primary amine groups with the carbonyl group in the polysaccharide by reductive amination. A primary amine group is used that is reactive with the carbonyl group in the polysaccharide. Hydrazide or hydroxylamino groups are suitable. The same primary amine group is typically present at both termini of the additional linker which allows for the possibility of polysaccharide (Ps)-Ps coupling. The reaction results in a polysaccharide-additional linker intermediate in which the polysaccharide is coupled to the additional linker via a C-N linkage.

Polysaccharide can be coupled to the additional linker using a different group in the polysaccharide, particularly a carboxyl group. This coupling comprises two steps: (a1) reacting the group with the additional linker; and (a2) reacting the free terminus of the additional linker with the linker. In this case, the additional linker typically has a primary amine group at both termini, thereby allowing step (a1) to take place by reacting one of the primary amine groups with the carboxyl group in the polysaccharide by EDAC activation. A primary amine group is used that is reactive with the EDAC-activated carboxyl group in the polysaccharide. A hydrazide group is suitable. The same primary amine group is typically present at both termini of the additional linker. The reaction results in a polysaccharide-additional linker intermediate in which the polysaccharide is coupled to the additional linker via an amide linkage.

### Carrier Protein

In a particular embodiment of the present invention, CRM197 is used as the carrier protein. CRM197 is anon-toxic variant (i.e., toxoid) of diphtheria toxin. CRM197 may be isolated from cultures of Corynebacterium diphtheria strain C7 (b197) grown in casamino acids and yeast extract-based medium. Further, CRM197 may be prepared recombinantly in accordance with the methods described in U.S. Pat. No. 5,614,382. Typically, CRM197 is purified through a combination of ultrafiltration, ammonium sulfate precipitation, and ion-exchange chromatography. In some embodiments, CRM197 is prepared in Pseudomonas fluorescens using Pfenex Expression Technology™ (Pfenex Inc., San Diego, CA).

Other suitable carrier proteins include additional inactivated bacterial toxins such as DT (Diphtheria toxoid), TT (tetanus toxoid) or fragment C of TT, pertussis toxoid, cholera toxoid (e.g., as described in International Patent Application Publication No. WO 2004/083251), E. coli LT, E. coli ST, and exotoxin A from Pseudomonas aeruginosa. Other suitable carrier proteins include CbpA and/or fractions of CbpA (pneumococcal choline binding protein A). Bacterial outer membrane proteins such as outer membrane complex c (OMPC), porins, transferrin binding proteins, pneumococcal surface protein A (PspA; See International Application Patent Publication No. WO 02/091998), pneumococcal surface adhesin protein (PsaA), C5a peptidase from Group A or Group B streptococcus, or Haemophilus influenzae protein D, pneumococcal pneumolysin (Kuo et al., 1995, Infect Immun 63; 2706-13) including ply detoxified in some fashion for example dPLY-GMBS (See International Patent Application Publication No. WO 04/081515) or dPLY-formol, PhtX, including PhtA, PhtB, PhtD, PhtE and fusions of Pht proteins for example PhtDE fusions, PhtBE fusions (See International Patent Application Publication Nos. WO 01/98334 and WO 03/54007), can also be used. Other proteins, such as ovalbumin, keyhole limpet hemocyanin (KLH), bovine serum albumin (BSA) or purified protein derivative of tuberculin (PPD), PorB (from N. meningitidis), PD (Haemophilus influenzae protein D; see, e.g., European Patent No. EP 0 594 610 B), or immunologically functional equivalents thereof, synthetic peptides (See European Patent Nos. EP0378881 and EP0427347), heat shock proteins (See International Patent Application Publication Nos. WO 93/17712 and WO 94/03208), pertussis proteins (See International Patent Application Publication No. WO 98/58668 and European Patent No. EP0471177), cytokines, lymphokines, growth factors or hormones (See International Patent Application Publication No. WO 91/01146), artificial proteins comprising multiple human CD4+ T cell epitopes from various pathogen derived antigens (See Falugi et al., 2001, Eur J Immunol 31:3816-3824) such as N19 protein (See Baraldoi et al., 2004, Infect Immun 72:4884-7), iron uptake proteins (See International Patent Application Publication No. WO 01/72337), toxin A or B of C. dificile (See International Patent Publication No. WO 00/61761), and flagellin (See Ben-Yedidia et al., 1998, Immunol Lett 64:9) can also be used as carrier proteins.

Where multivalent vaccines are used, a second carrier can be used for one or more of the antigens in a multivalent vaccine. The second carrier protein is preferably a protein that is non-toxic and non-reactogenic and obtainable in sufficient amount and purity. The second carrier protein is also conjugated or joined with an antigen, e.g., a S. pneumoniae polysaccharide to enhance immunogenicity of the antigen. Carrier proteins should be amenable to standard conjugation procedures. Each capsular polysaccharide not conjugated to a first carrier protein may be conjugated to the same second carrier protein (e.g., each capsular polysaccharide molecule being conjugated to a single carrier protein). Capsular polysaccharides not conjugated to a first carrier protein may be conjugated to two or more carrier proteins (each capsular polysaccharide molecule being conjugated to a single carrier protein). In such embodiments, each capsular polysaccharide of the same serotype is typically conjugated to the same carrier protein. Other DT mutants can be used as the second carrier protein, such as CRM176, CRM228, CRM45 (Uchida et al., 1973, J Biol Chem 218:3838-3844); CRM9, CRM45 CRM102, CRM103 and CRM107 and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. No. 4,709,017 or U.S. Pat. No. 4,950,740; mutation of at least one or more residues Lys 516, Lys 526, Phe 530 and/or Lys 534 and other mutations disclosed in U.S. Pat. No. 5,917,017 or U.S. Pat. No. 6,455,673; or fragment disclosed in U.S. Pat. No. 5,843,711.

### Conjugation by Reductive Amination

Covalent coupling of polysaccharide to carrier protein can be performed via reductive amination in which an amine-reactive moiety on the polysaccharide is directly coupled to primary amine groups (mainly lysine residues) of the protein. As is well known, a reductive amination reaction proceeds via a two-step mechanism. First, a Schiff base intermediate, of formula R—CH═N—R′, is formed by reaction of an aldehyde group on molecule 1 (R-CHO) with a primary amine group (R′-NH2) on molecule 2. In the second step, the Schiff base is reduced to form an amino compound of formula R-CH2-NH-R′. While many reducing agents are capable of being utilized, most often a highly selective reducing agent such as sodium cyanoborohydride (NaCNBH₃) is employed since such reagents will specifically reduce only the imine function of the Schiff base.

Since all the polysaccharides have an aldehyde function at the end of the chain (terminal aldehyde function), the conjugation methods comprising a reductive amination of the polysaccharide can be applied very generally and, when there is no other aldehyde function in the repeating unit (intrachain aldehyde function), such methods make it possible to obtain conjugates in which a polysaccharide molecule is coupled to a single molecule of carrier protein.

A typical reducing agent is cyanoborohydride salt such as sodium cyanoborohydride. The imine-selective reducing agent typically employed is sodium cyanoborohydride, although other cyanoborohydride salts can be used including potassium cyanoborohydride. Differences in starting cyanide levels in sodium cyanoborohydride reagent lots and residual cyanide in the conjugation reaction can lead to inconsistent conjugation performance, resulting in variable product attributes, such as conjugate size and conjugate Ps-to-CRM197 ratio. By controlling and/or reducing the free cyanide levels in the final reaction product, conjugation variability can be reduced.

Residual unreacted aldehydes on the polysaccharide are optionally reduced with the addition of a strong reducing agent, such as sodium borohydride. Generally, use of a strong reducing agent is preferred. However, for some polysaccharides, it is preferred to avoid this step. For example, S. pneumoniae serotype 5 contains a ketone group that may react readily with a strong reductant. In this case, it is preferable to bypass the reduction step to protect the antigenic structure of the polysaccharide.

Following conjugation, the polysaccharide-protein conjugates are purified to remove excess conjugation reagents as well as residual free protein and free polysaccharide by one or more of any techniques well known to the skilled artisan, including concentration/diafiltration operations, ultrafiltration, precipitation/elution, column chromatography, and depth filtration. See, e.g., U.S. Pat. No. 6,146,902. In one embodiment, the purifying step is by ultrafiltration.

### Pneumococcal Conjugate Compositions

The present invention provides pneumococcal conjugate compositions comprising, consisting essentially of, or alternatively, consisting of polysaccharide-protein conjugates together with an LNP adjuvant. The present invention further provides pneumococcal conjugate compositions comprising, consisting essentially of, or alternatively, consisting of polysaccharide-protein conjugates together with an LNP adjuvant and a pharmaceutically acceptable carrier. The present invention further provides pneumococcal conjugate compositions comprising, consisting essentially of, or alternatively, consisting of any of the polysaccharide-protein conjugate combinations described herein together with an LNP and optionally a pharmaceutically acceptable carrier. The compositions of the instant invention may comprise, consist essentially of, or consist of 2 to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 distinct polysaccharide-protein conjugates, wherein each of the conjugates contains a different capsular polysaccharide conjugated to a carrier protein. In an embodiment, the polysaccharides that are a part of the polysaccharide-protein conjugates are selected from at least one of serotypes 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20, 20A, 20B, 22F, 23A, 23B, 23F, 24F, 33F, 35B, 35F, or 38 of Streptococcus pneumoniae. In another embodiment, the group of serotypes comprise, consist essentially of, or consist of 4, 6B, 9V, 14, 18C, 19F and 23F. In another embodiment, the group of serotypes comprise, consist essentially of, or consist of 1, 3, 4, 5, 6A, 6B, 7F, 9V, 14, 18C, 19A, 19F and 23F. In another embodiment, the group of serotypes comprise, consist essentially of, or consist of 1, 3, 4, 5, 6A, 6B, 7F, 9V, 14, 18C, 19A, 19F, 22F, 23F and 33F. In another embodiment, the group of serotypes comprise, consist essentially of, or consist of 1, 3, 4, 5, 6A, 6B, 7F, 8, 9V, 10A, IA, 12F, 14, 15B, 18C, 19A, 19F, 22F, 23F and 33F. In another embodiment, the group of serotypes comprise, consist essentially of, or consist of 1, 3, 4, 5, 6A, 6B, 7F, 8, 9V, 10A, IA, 12F, 14, 15A, 15B, 18C, 19A, 19F, 22F, 23B, 23F, 24F, 33F and 35B. In another embodiment, the group of serotypes comprise, consist essentially of, or consist of 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B. In another embodiment, the group of serotypes comprise, consist essentially of, or consist of 1, 3, 4, 5, 6A, 6B, 7F, 8, 9V, 10A, IA, 12F, 14, 15A, 15C, 18C, 19A, 19F, 22F, 23B, 23F, 24F, 33F and 35B. In another embodiment the group of serotypes comprise, consist essentially of, or consist of 3, 6A, 7F, 8, 9N, 10A, IA, 12F, 15A, 15B, 16F, 17F, 19A, 20A, 22F, 23A, 23B, 24F, 31, 33F and 35B. In another embodiment the group of serotypes comprise, consist essentially of, or consist of 3, 6A, 7F, 8, 9N, 10A, IA, 12F, 15A, de-O-acetylated-15B, 16F, 17F, 19A, 20A, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, 15C, 16F, 17F, 19A, 20A, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, 15B, 16F, 17F, 19A, 20, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, de-O-acetylated-15B, 16F, 17F, 19A, 20, 22F, 23A, 23B, 24F, 31, 33F and 35B. In another embodiment the group of serotypes comprise, consist essentially of, or consist of 3, 6A, 7F, 8, 9N, 10A, IA, 12F, 15A, 15C, 16F, 17F, 19A, 20, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, 15B, 16F, 17F, 19A, 20B, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, de-O-acetylated-15B, 16F, 17F, 19A, 20B, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, 15C, 16F, 17F, 19A, 20B, 22F, 23A, 23B, 24F, 31, 33F and 35B. In another embodiment the serotypes are conjugated to the carrier protein CRM197.

### Administration/Dosage

The compositions of the present invention can be used to protect or treat a human susceptible to infection, e.g., a pneumococcal infection, by means of administering the vaccine via a systemic or mucosal route. In one embodiment, the present invention provides a method of inducing an immune response to a S. pneumoniae capsular polysaccharide conjugate, comprising administering to a human an immunologically effective amount of a composition of the present invention. In another embodiment, the present invention provides a method of vaccinating a human against a pneumococcal infection, comprising the step of administering to the human an immunologically effective amount of a composition of the present invention.

Optimal amounts of components for a particular composition can be ascertained by standard studies involving observation of appropriate immune responses in subjects. For example, in another embodiment, the dosage for human vaccination is determined by extrapolation from animal studies to human data. In another embodiment, the dosage is determined empirically.

The methods of the invention can be used for the prevention and/or reduction of primary clinical syndromes caused by S. pneumonia, including both invasive infections (meningitis, pneumonia, and bacteremia), and noninvasive infections (acute otitis media, and sinusitis).

Administration of the compositions of the invention can include one or more of: injection via the intramuscular, intraperitoneal, intradermal or subcutaneous routes; or via mucosal administration to the oral/alimentary, respiratory or genitourinary tracts. In one embodiment, intranasal administration is used for the treatment of pneumonia or otitis media (as nasopharyngeal carriage of pneumococci can be more effectively prevented, thus attenuating infection at its earliest stage).

The amount of conjugate in each dose may be selected as an amount that induces an immunoprotective response without significant, adverse effects. Such amount can vary depending upon the pneumococcal serotype. Generally, for polysaccharide-based conjugates, each dose will comprise 0.1 to 100 mg of each polysaccharide serotype, particularly 0.1 to 10 μg, and more particularly 1 to 8 μg per serotype. For example, each dose can comprise 100, 150, 200, 250, 300, 400, 500, or 750 ng or 1, 1.5, 2, 3, 4, 5, 6, 7, 7.5, 8, 9, 10, 11, 12, 13, 14, 15, 16, 18, 20, 22, 25, 30, 40, 50, 60, 70, 80, 90, or 100 μg of each polysaccharide serotype.

According to any of the methods of the present invention and in one embodiment, the subject is human. In certain embodiments, the human patient is an infant (less than 1 year of age), toddler (approximately 12 to 24 months), or young child (approximately 2 to 5 years). In other embodiments, the human patient is an elderly patient (>65 years). The compositions of this invention are also suitable for use with older children, adolescents and adults (e.g., aged 18 to 45 years or 18 to 65 years).

In one embodiment of the methods of the present invention, a composition of the present invention is administered as a single inoculation. In another embodiment, the vaccine is administered two, three, four or more times, adequately spaced apart. For example, the composition may be administered at 1-, 2-, 3-, 4-, 5-, or 6-month intervals or any combination thereof. The immunization schedule can follow that designated for pneumococcal vaccines. For example, the routine schedule for infants and toddlers against invasive disease caused by S. pneumoniae is 2-, 4-, 6- and 12-15-months of age. Thus, in a preferred embodiment, the composition is administered as a 4-dose series at 2-, 4-, 6-, and 12-15-months of age.

The compositions of this invention may also include one or more proteins from S. pneumoniae. Examples of S. pneumoniae proteins suitable for inclusion include those identified in International Patent Application Publication Nos. WO 02/083855 and WO 02/053761.

### Inventive Formulations

In some embodiments, a composition is provided that includes an LNP and S. pneumoniae polysaccharide-protein conjugates. In some embodiments, a composition is provided that includes an LNP and S. pneumoniae polysaccharide-protein conjugates containing at least 1, or at least 3, or at least 7, or at least 10, or at least 13, or at least 15, or at least 20, or at least 24, or at least 27, or at least 30 S. pneumoniae serotypes. In some embodiments, a composition is provided that includes an LNP and S. pneumoniae polysaccharide-protein conjugates containing 20, 21, 22, 23, 24, 25, 26, 27, 28, 29, 30, 31, 32, 33, 34, 35, 36, 37, 38, 39 or 40 S. pneumoniae serotypes. In some embodiments, a composition is provided that includes an LNP and S. pneumoniae polysaccharide-protein conjugates containing S. pneumoniae serotypes consisting of 4, 6B, 9V, 14, 18C, 19F and 23F. In some embodiments, a composition is provided that includes an LNP and S. pneumoniae polysaccharide-protein conjugates containing S. pneumoniae serotypes consisting of 1, 3, 4, 5, 6A, 6B, 7F, 9V, 14, 18C, 19A, 19F and 23F. In some embodiments, a composition is provided that includes an LNP and S. pneumoniae polysaccharide-protein conjugates containing S. pneumoniae serotypes consisting of 1, 3, 4, 5, 6A, 6B, 7F, 9V, 14, 18C, 19A, 19F, 22F, 23F and 33F. In some embodiments, a composition is provided that includes an LNP and S. pneumoniae polysaccharide-protein conjugates containing S. pneumoniae serotypes consisting of 1, 3, 4, 5, 6A, 6B, 7F, 8, 9V, 10A, 11A, 12F, 14, 15B, 18C, 19A, 19F, 22F, 23F and 33F. In some embodiments, a composition is provided that includes an LNP and S. pneumoniae polysaccharide-protein conjugates containing S. pneumoniae serotypes consisting of 1, 3, 4, 5, 6A, 6B, 7F, 9V, 10A, 12F, 14, 15A, 15B, 18C, 19A, 19F, 22F, 23B, 23F, 24F, 33F and 35B. In some embodiments, a composition is provided that includes an LNP and S. pneumoniae polysaccharide-protein conjugates containing S. pneumoniae serotypes consisting of 1, 3, 4, 5, 6A, 6B, 7F, 9V, 10A, 12F, 14, 15A, de-O-acetyl-15B, 18C, 19A, 19F, 22F, 23B, 23F, 24F, 33F and 35B. In some embodiments, a composition is provided that includes an LNP and S. pneumoniae polysaccharide-protein conjugates containing S. pneumoniae serotypes consisting of 1, 3, 4, 5, 6A, 6B, 7F, 9V, 10A, 12F, 14, 15A, 15C, 18C, 19A, 19F, 22F, 23B, 23F, 24F, 33F and 35B. In some embodiments, a composition is provided that includes an LNP and S. pneumoniae polysaccharide-protein conjugates containing S. pneumoniae serotypes consisting of 1, 3, 4, 5, 6A, 6B, 7F, 9V, 10A, 12F, 14, 15A, 15B, 18C, 19A, 19F, 22F, 23B, 23F, 24F, 33F and 35B. In some embodiments, a composition is provided that includes an LNP and S. pneumoniae polysaccharide-protein conjugates containing S. pneumoniae serotypes consisting of 1, 3, 4, 5, 6A, 6B, 7F, 9V, 10A, 12F, 14, 15A, de-O-acetyl-15B, 18C, 19A, 19F, 22F, 23B, 23F, 24F, 33F and 35B. In some embodiments, a composition is provided that includes an LNP and S. pneumoniae polysaccharide-protein conjugates containing S. pneumoniae serotypes consisting of 1, 3, 4, 5, 6A, 6B, 7F, 9V, 10A, 12F, 14, 15A, 15C, 18C, 19A, 19F, 22F, 23B, 23F, 24F, 33F and 35B. In some embodiments, a composition is provided that includes an LNP and S. pneumoniae polysaccharide-protein conjugates containing S. pneumoniae serotypes consisting of 1, 3, 4, 5, 6A, 6B, 7F, 8, 9V, 10A, 11A, 12F, 14, 15A, 15B, 18C, 19A, 19F, 22F, 23B, 23F, 24F, 33F and 35B. In some embodiments, a composition is provided that includes an LNP and S. pneumoniae polysaccharide-protein conjugates containing S. pneumoniae serotypes consisting of 1, 3, 4, 5, 6A, 6B, 7F, 8, 9V, 10A, 11A, 12F, 14, 15A, de-0-acetyl-15B, 18C, 19A, 19F, 22F, 23B, 23F, 24F, 33F and 35B. In some embodiments, a composition is provided that includes an LNP and S. pneumoniae polysaccharide-protein conjugates containing S. pneumoniae serotypes consisting of 1, 3, 4, 5, 6A, 6B, 7F, 8, 9V, 10A, 11A, 12F, 14, 15A, 15C, 18C, 19A, 19F, 22F, 23B, 23F, 24F, 33F and 35B. In some embodiments, a composition is provided that includes an LNP and S. pneumoniae polysaccharide-protein conjugates containing S. pneumoniae serotypes consisting of 3, 6A, 7F, 8, 9N, 10A, IA, 12F, 15A, 15B, 16F, 17F, 19A, 20A, 22F, 23A, 23B, 24F, 31, 33F and 35B. In some embodiments, a composition is provided that includes an LNP and S. pneumoniae polysaccharide-protein conjugates containing S. pneumoniae serotypes consisting of 3, 6A, 7F, 8, 9N, 10A, IA, 12F, 15A, de-O-acetylated-15B, 16F, 17F, 19A, 20A, 22F, 23A, 23B, 24F, 31, 33F and 35B. In some embodiments, a composition is provided that includes an LNP and S. pneumoniae polysaccharide-protein conjugates containing S. pneumoniae serotypes consisting of 3, 6A, 7F, 8, 9N, 10A, IA, 12F, 15A, 15C, 16F, 17F, 19A, 20A, 22F, 23A, 23B, 24F, 31, 33F and 35B. In some embodiments, a composition is provided that includes an LNP and S. pneumoniae polysaccharide-protein conjugates containing S. pneumoniae serotypes consisting of 3, 6A, 7F, 8, 9N, 10A, IA, 12F, 15A, 15B, 16F, 17F, 19A, 20, 22F, 23A, 23B, 24F, 31, 33F and 35B. In some embodiments, a composition is provided that includes an LNP and S. pneumoniae polysaccharide-protein conjugates containing S. pneumoniae serotypes consisting of 3, 6A, 7F, 8, 9N, 10A, IA, 12F, 15A, de-O-acetylated-15B, 16F, 17F, 19A, 20, 22F, 23A, 23B, 24F, 31, 33F and 35B. In some embodiments, a composition is provided that includes an LNP and S. pneumoniae polysaccharide-protein conjugates containing S. pneumoniae serotypes consisting of 3, 6A, 7F, 8, 9N, 10A, IA, 12F, 15A, 15C, 16F, 17F, 19A, 20, 22F, 23A, 23B, 24F, 31, 33F and 35B. In some embodiments, a composition is provided that includes an LNP and S. pneumoniae polysaccharide-protein conjugates containing S. pneumoniae serotypes consisting of 3, 6A, 7F, 8, 9N, 10A, IA, 12F, 15A, 15B, 16F, 17F, 19A, 20B, 22F, 23A, 23B, 24F, 31, 33F and 35B. In some embodiments, a composition is provided that includes an LNP and S. pneumoniae polysaccharide-protein conjugates containing S. pneumoniae serotypes consisting of 3, 6A, 7F, 8, 9N, 10A, IA, 12F, 15A, de-O-acetylated-15B, 16F, 17F, 19A, 20B, 22F, 23A, 23B, 24F, 31, 33F and 35B. In some embodiments, a composition is provided that includes an LNP and S. pneumoniae polysaccharide-protein conjugates containing S. pneumoniae serotypes consisting of 3, 6A, 7F, 8, 9N, 10A, 11A, 12F, 15A, 15C, 16F, 17F, 19A, 20B, 22F, 23A, 23B, 24F, 31, 33F and 35B.

In an embodiment of the compositions above, the compositions do not comprise polysaccharide-protein conjugates containing polysaccharides of any other S. pneumoniae serotype.

In some embodiments, the compositions described above can contain an additional 1, 2, 3, 4 or 5 S. pneumoniae polysaccharide-protein conjugates containing other S. pneumoniae serotypes known in the art.

In some embodiments, the compositions described above comprise polysaccharide-protein conjugates wherein the protein is the carrier protein CRM197.

In some embodiments, a composition is provided that includes about 1 μg to about 200 mg LNP and at least one S. pneumoniae polysaccharide-protein conjugate, wherein each of the conjugates is present in a concentration of about 0.01 μg to about 100 μg per 0.5 mL of the composition.

In some embodiments, a composition is provided that includes about 0.02 μg to about 40 mg LNP and at least one S. pneumoniae polysaccharide-protein conjugate, wherein each of the conjugates is present in a concentration of about 0.002 μg to about 20 μg per 0.1 mL of the composition.

In some embodiments, a composition is provided that includes about 50 μg to about 2.1 mg LNP and at least one S. pneumoniae polysaccharide-protein conjugate, wherein each of the conjugates is present in a concentration of about 0.002 μg to about 20 μg per 0.5 mL of the composition.

In some embodiments, a composition is provided that includes about 50 μg to about 10 mg LNP and at least one S. pneumoniae polysaccharide-protein conjugate, wherein each of the conjugates is present in a concentration of about 0.002 μg to about 20 μg per 0.5 mL of the composition.

In some embodiments, a composition is provided that includes about 1 μg to about 200 mg LNP and at least one S. pneumoniae polysaccharide-protein conjugate, wherein each of the conjugates is present in a concentration of about 0.01 μg to about 100 μg per 0.5 mL of the composition prepared as a co-lyophilized formulation.

In some embodiments, a composition is provided that includes about 1 μg to about 200 mg LNP, 1 μg to about 1 mg of aluminum in the form of APA and at least one S. pneumoniae polysaccharide-protein conjugate wherein the conjugates containing any one S. pneumoniae serotype are present in a concentration of about 0.01 μg to about 100 μg per 0.5 mL of the composition prepared as a co-lyophilized formulation.

In some embodiments, a composition, as highlighted in the various embodiments above, is provided that includes about 0.05 μg to about 200 mg LNP which is composed of (1) a cationic lipid, (2) cholesterol, (3) a phospholipid, DSPC, and (4) a PEG-lipid (ePEG2000-DMG).

In another embodiment, the cationic lipid is CLA. In another embodiment, the cationic lipid is CLX. In another embodiment, the cationic lipid is CLY.

Compositions of the present invention may be administered subcutaneously, topically, orally, on the mucosa, intravenously, or intramuscularly. The compositions are administered in an amount sufficient to elicit a protective response. Compositions can be administered by various routes, for example, orally, parenterally, subcutaneously, on the mucosa, or intramuscularly. The dose administered may vary depending on the general condition, sex, weight and age of the patient, and the route of administration.

Compositions of the present invention, as highlighted in the various embodiments above, may be referred to as immunogenic compositions.

Compositions of the present invention, as highlighted in the various embodiments above, may be referred to a vaccines or vaccine compositions.

In an embodiment, the composition of any of the embodiments described above is provided, wherein the LNP comprises 30-65 mole % cationic lipid, 5-30 mole % phospholipid, 10-40% cholesterol, and 0.5-4 mole % PEG-lipid.

In an embodiment, the composition of any of the embodiments described above is provided, wherein the LNP comprises 55-65 mole % cationic lipid, 5-15 mole % phospholipid, 25-35% cholesterol, and 1-2.5 mole % PEG-lipid.

In an embodiment, the composition of any of the embodiments described above is provided, wherein the LNP comprises DSPC, cholesterol, PEG2000-DMG, and (13Z,16Z)-N, N-dimethyl-3-nonyldocosa 13, 16-dien-1-amine.

In an embodiment, the composition of any of the embodiments described above is provided, wherein the LNP comprises 5-15 mole % DSPC, 25-35 mole % cholesterol, 1-2.5 mol % PEG2000-DMG, and 55-65 mole % (13Z,16Z)-N,N-dimethyl-3-nonyldocosal3,16-dien-1-amine.

In an embodiment, the composition of any of the embodiments described above is provided, wherein the LNP comprises DSPC, cholesterol, ePEG2000-DMG, and (13Z,16Z)-N, N-dimethyl-3-nonyldocosa 13, 16-dien-1-amine.

In an embodiment, the composition of any of the embodiments described above is provided, wherein the LNP comprises 5-15 mole % DSPC, 25-35 mole % cholesterol, 1-2.5 mol % ePEG2000-DMG, and 55-65 mole % (13Z,16Z)-N,N-dimethyl-3-nonyldocosal3,16-dien-1-amine.

All publications mentioned herein are incorporated by-reference for the purpose of describing and disclosing methodologies and materials that might be used in connection with the present invention. Nothing herein is to be construed as an admission that the invention is not entitled to antedate such disclosure by virtue of prior invention.

Having described preferred embodiments of the invention with reference to the accompanying drawings, it is to be understood that the invention is not limited to those precise embodiments, and that various changes and modifications may be used by one skilled in the art without departing from the scope or spirit of the invention as defined in the appended claims.

The following examples illustrate, but do not limit the invention.

### EXAMPLES

### Example 1: Preparation of Pneumococcal Polysaccharide-Protein Conjugates Using DMSO Conjugation

Polysaccharide(s) (as highlighted below and in the Tables and Examples) was dissolved, sized to a target molecular mass, chemically activated and buffer-exchanged by ultrafiltration. Activated polysaccharide and purified CRM197 were individually lyophilized and re-dissolved in DMSO. Re-dissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction

Purified pneumococcal capsular polysaccharide (otherwise termed “Ps”) powder was dissolved in water. With the exception of ST-19A, (serotype is otherwise termed “ST”) which is not sized reduced, dissolved polysaccharide was 0.45-micron filtered and either homogenized or acid hydrolyzed to reduce the molecular mass of the Ps. Target Ps size was achieved for homogenization by controlling the pressure and number of passes. Target Ps size was achieved for acid hydrolysis by controlling the temperature and time. Polysaccharide was then 0.2-micron filtered and concentrated and diafiltered against water using a 5 or 10 kDa NMWCO tangential flow ultrafiltration membrane.

### De-O-acetylation

Size reduced ST-15B Ps solution was heated to 60° C. and sodium bicarbonate pH 9.4 buffer was added to a final concentration of 50 mM. The batch was incubated at 60° C. to release O-acetyl groups. Potassium phosphate pH 6 buffer was added to neutralize pH and the solution was cooled to ambient temperature. The solution was then concentrated and diafiltered against water using a 5 or 10 kDa NMWCO tangential flow ultrafiltration membrane.

### Deketalization (ST-4 Only)

Sized reduced ST-4 Ps solution was adjusted to 50° C. and pH 4.1 with a sodium acetate buffer to partially deketalize the polysaccharide. The polysaccharide solution was then cooled to 22° C. prior to activation.

### Polysaccharide Oxidation

The polysaccharide solution was adjusted to 22° C. for all serotypes, except for ST-5, 7F and 19F, which were adjusted to 4° C. The solution was also adjusted to pH 4-5 with a sodium acetate buffer to minimize polysaccharide size reduction due to activation. Polysaccharide activation was initiated with the addition of a sodium metaperiodate solution. The amount of sodium metaperiodate added was controlled to achieve a target level of polysaccharide activation (moles aldehyde per mole of polysaccharide repeating unit).

The activated product for all serotypes except ST-5 was diafiltered against 10 mM potassium phosphate, pH 6.4 followed by diafiltration against water using a 5 or 10 kDa NMWCO tangential flow ultrafiltration membrane. For ST-5, the activated product was diafiltered against 10 mM sodium acetate, pH 4.1 followed by diafiltration against water using a 5 kDa NMWCO tangential flow ultrafiltration membrane. Ultrafiltration was conducted at 2-8° C. for all serotypes.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered. Activated polysaccharides were formulated for lyophilization with water and sucrose. CRM197 was formulated for lyophilization at 6 mg Pr/mL (the CRM197 protein is otherwise referred to as “Pr”) with sucrose concentration of 1% w/v. Formulated Ps and CRM197 solutions were individually lyophilized. Lyophilized Ps and CRM197 materials were re-dissolved individually in equal volumes of DMSO. Additives such as salt were spiked into the Ps-DMSO for some serotypes. The polysaccharide and CRM197 solutions were blended to achieve a target polysaccharide concentration and polysaccharide to CRM197 mass ratio. The mass ratio was selected to control the polysaccharide to CRM197 ratio in the resulting conjugate. A reducing agent such as sodium cyanoborohydride was added for most serotypes and conjugation proceeded at 22° C.

### Final Reduction

A reducing agent such as sodium borohydride was added following the conjugation reaction and incubated at 22° C. for all serotypes. The batch was diluted into 150 mM sodium chloride, with approximately 0.025% (w/v) polysorbate 20, at approximately 4° C. Potassium phosphate buffer was then added to neutralize the pH. Some lots were concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

Individual batches were then concentrated and diafiltered against 10 mM histidine in 150 mM sodium chloride, pH 7.0, with 0.015% (w/v) polysorbate 20, at 4° C. using a 300 kDa NMWCO tangential flow ultrafiltration membrane. Specifically, for ST-5, halfway through the diafiltration step, ST-5 conjugate was harvested and incubated with 50 mM sodium bicarbonate, pH 9.3 for 3 hours. The ST-5 solution was neutralized with 1.5 M potassium phosphate, pH 6.0 prior to completing diafiltration.

The individual retentate batches were 0.2-micron filtered (with 0.5-micron prefilter) then diluted with additional 10 mM histidine in 150 mM sodium chloride, pH 7.0 with 0.015% (w/v) polysorbate 20, dispensed into aliquots and frozen at <−60° C. Serotype specific conjugate details can be found as previously described (WO2011/100151, WO2019/139692 and WO2020/131763).

### Example 2: Formulation of Pneumococcal Conjugate Compositions

Individual pneumococcal polysaccharide-protein conjugates prepared utilizing different chemistries as described in Example 1 were used for the formulation of a 1-, 22- and 24-valent pneumococcal conjugate vaccine composition referred to as PCV1, PCV22 and PCV24, respectively. Individual pneumococcal polysaccharides as described in Example 1, were used for the formulation of a 22-valent pneumococcal polysaccharide vaccine referred to as PPSV22.

The PCV1 formulation, prepared with or to be added to the CL #-LNP (whereby CL #can be CLA, CLX or CLY) contains serotype 4 (ST-4) conjugated using reductive amination in an aprotic (DMSO) solvent, as described in Example 1, and formulated in 20 mM L-Histidine pH 5.8, 150 mM NaCl and 0.1% (w/v) PS-20 for a final concentration of 0.8 μg/mL (w/v) pneumococcal polysaccharide (also referred to as PnPs) in the vaccine composition. The PCV1 vaccine formulation prepared with Aluminum Phosphate Adjuvant (APA) and serotype 4 (ST-4) conjugated using reductive amination in an aprotic (DMSO) solvent, as described in Example 1, was formulated in 20 mM L-Histidine pH 5.8, 150 mM NaCl and 0.2% (w/v) PS-20 and 50 μg [Al⁺³]/mL in the form of Aluminum Phosphate Adjuvant (APA) for a final concentration of 0.8 μg/mL (w/v) pneumococcal polysaccharide (PnPs) in the vaccine composition as described in the specific Example, below.

The PCV22 composition contains serotypes 1, 3, 4, 5, 6A, 6B, 7F, 9V, 10A, 12F, 14, 15A, de-O-Ac-15B, 18C, 19A, 19F, 22F, 23B, 23F, 24F, 33F and 35B conjugated using reductive amination in an aprotic solvent (e.g. DMSO) and formulated in 20 mM L-Histidine pH 5.8 150 mM NaCl and 0.1% PS-20. Each polysaccharide-protein conjugate was formulated at 0.8 μg/mL (w/v) pneumococcal polysaccharide (PnPs) for a final concentration of 17.6 μg/mL PnPs in the vaccine. The PCV22 vaccine formulation prepared with Aluminum Phosphate Adjuvant (APA) was formulated in 20 mM L-Histidine pH 5.8, 150 mM NaCl and 0.2% (w/v) PS-20 and 50 or 1250 μg [Al⁺³]/mL in the form of Aluminum Phosphate Adjuvant (APA) for a final concentration of 0.8 μg/mL (w/v) pneumococcal polysaccharide (PnPs) per serotype or 17.6 μg/mL in the vaccine composition as described in the specific Example, below.

A PPSV22 composition containing serotypes 1, 3, 4, 5, 6A, 6B, 7F, 9V, 10A, 12F, 14, 15A, de-O-Ac-15B, 18C, 19A, 19F, 22F, 23B, 23F, 24F, 33F and 35B was formulated in 20 mM L-Histidine pH 5.8 150 mM NaCl and 0.1% PS-20. Each polysaccharide-protein conjugate was formulated at 0.8 μg/mL (w/v) pneumococcal polysaccharide (PnPs) for a final concentration of 17.6 μg/mL PnPs in the vaccine.

The PCV24 composition contains serotypes 1, 3, 4, 5, 6A, 6B, 7F, 8, 9V, 10A, 11A, 12F, 14, 15A, de-O-Ac-15B, 18C, 19A, 19F, 22F, 23B, 23F, 24F, 33F and 35B conjugated using reductive amination in an aprotic solvent (e.g. DMSO) and formulated in 20 mM L-Histidine pH 5.8 150 mM NaCl and 0.1% PS-20. Each polysaccharide-protein conjugate was formulated at 4 to 8 μg/mL (w/v) pneumococcal polysaccharide (PnPs) for a final concentration of 96 to 192 μg/mL PnPs in the vaccine.

To prepare the PCV formulations, the required volumes of monovalent bulk conjugates needed to obtain the indicated final concentration of (w/v) pneumococcal polysaccharide (PnPs) were calculated based on the batch volume and the bulk polysaccharide concentration.

The formulation process consisted of a conjugate bulk blend preparation at 2× the final concentration of PnPs blends in 20 mM Histidine, 0.05 to 0.15% (w/v) PS-20, and 150 mM sodium chloride, pH 5.8.

Histidine pH 5.8, PS-20 and sodium chloride solutions were prepared and added to the formulation vessel. The individual pneumococcal polysaccharide-protein conjugates, stored frozen, were thawed at 2-8° C. and then added to the formulation vessel. During the addition of polysaccharide-protein conjugate to the formulation buffer (conjugate blend), the vessel was mixed to ensure homogeneity using a magnetic sir bar or magnetic impeller. After all additions were made and the solution stirred, the conjugate blend was passed through sterilizing filters and collected in a vessel with or without APA. In some cases, the sterilizing filters were chased with 150 mM sodium chloride to adjust the batch to target concentration.

The formulations were filled into plastic syringes, glass syringes, or vials.

### Example 3: Preparation of the LNP

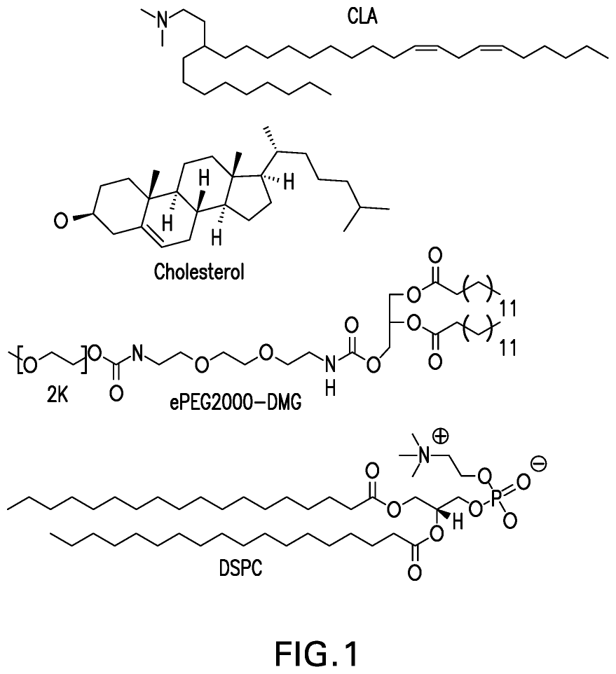

The adjuvant of the instant invention is a multi-component LNP formulation consisting of 4 components; one cationic lipid (referred to as CL #), cholesterol, distearoyl phosphatidyl choline (DSPC), and ePEG2000-DMG (FIG. 1, shown with preferred cationic lipid, CLA, (13Z,16Z)-N,N-dimethyl-3-nonyldocosa-13,16-dien-1-amine).

The final CL #-LNP formulation relative target mole % values for the lipid components are 58% CL #, 30% cholesterol, 2% ePEG2000-DMG and 10% DSPC (Table 1).

TABLE 1
Composition of CL#-LNP Adjuvant

| | | Content of | Molecular | Content of |
|---|---|---|---|---|
| | | Each Lipid | Weight | Each Lipid |
| Component | Description | (Mole %) | (g/mol) | (Mass %) |
| CL# | Cationic Lipid | 58 | 475.9 | 52 |
| Cholesterol | Cholest-5-en-3β-ol | 30 | 386.7 | 22 |
| Distearoyl | 1,2-distearoyl-sn-glycero-3- | 10 | 790.2 | 15 |
| Phosphitidyl | phosphocholine | | | |
| Choline (DSPC) | | | | |
| ePEG2000-DMG | α-[8′-(1,2-Dimyristoyl-3- | 2 | 2837 | 11 |
| | propanoxy)-carboxamide-3′, | | | |
| | 6′-Dioxaoctanyl]carbamoyl-ω- | | | |
| | methyl-poly(ethylene glycol)- | | | |
| | 2000 | | | |
| Buffer Matrix | 20 mM Tris 10% (w/v) Sucrose | N/A | | |
| | pH 7.5 | | | |

As described below, the process of preparing CLA #-LNP bulk adjuvant consists of 5 steps: 1) solution preparation of lipid mixture and diluted citrate A; 2) LNP formation by means of T-mixing; 3) ultra-filtration; 4) bioburden reduced filtration; and 5) sterile filtration and vial filling.

### Solution Preparation of Lipid Mixture and Diluted Citrate A

The lipid components were weighed and combined before being dissolved in ethanol before being sterile filtered to form the lipid mixture. Citrate A (20 mM Citrate pH 5.0) was diluted at a one-to-one ratio with sterile water to form diluted citrate A (DCA).

### LNP Formation by Means of T-Mixing

The lipid mixture and DCA are then mixed at adjacent ends of a T-tube mixer. The stream exiting the T-mix apparatus is immediately diluted 1:1 with 20 mM citrate, 300 mM NaCl pH 6.0, this product mixture is again diluted 1:1 with 1× Dulbecco's phosphate buffered saline and is then collected as formed LNP. The LNP intermediate is then incubated at ambient temperature for 30 minutes before being held overnight at 4° C.

### Ultra-Filtration

The LNP intermediate is then subjected to ultra-filtration with a 500 kDA NMWCO to both concentrate the material approximately 10-fold as well as diafilter the material against 20 mM Tris, 10% (w/v) sucrose, pH 7.5. After the diafiltration, there is a final concentration step performed to achieve final target concentration.

### Bioburden Reduced Filtration

The adjuvant bulk is then pre-filtered with a 0.45 μm cellulose acetate (CA) filter followed by a 0.2 μm CA bioburden-reducing filter, and stored frozen at −70° C.

### Sterile Filtration and Vial Filling

Frozen LNP bulk is thawed in a 25+/−3° C. controlled water bath. The thawed LNP bulk is passed through a 0.45 μm polyvinylidene fluoride (PVDF) bioburden reducing filter and a 0.22 μm PVDF sterilizing grad filter and received. The filtered LNP bulk is then diluted with 20 mM Tris, 10% (w/v) sucrose, pH 7.5 to the target LNP concentration. This diluted final LNP is then filled into glass vials and stored at −70° C.

### Example 4: Preparation of a PCV24 and CL #-LNP Lyophilized Formulation

A PCV24 composition was prepared as described in Example 2 and combined with the CLA-LNP bulk, prepared as described in Example 3. The PCV24/CLA-LNP formulation stabilized in 10 mM L-histidine, 10 mM Tris, 75 mM NaCl, 0.05% w/v PS-20 pH 6.2 was used to prepare a lyophilized cake. For preparation of the PCV24/CL #-LNP lyophilized cake, the formulations are frozen on a −50° C. pre-cooled shelf. The vacuum set point for this lyophilization cycle remained at 50 mTorr. Thermal treatment, or a step designed to remove heat from the formulation material, was then completed at −50° C. for 2 hr. at atmospheric pressure (1 atm). After the thermal treatment, there is an intermittent pressure step that descends to 50 mTorr for 1 minute with shelf temperature setpoint held at −50° C. to ensure functional vacuum control of the lyophilization unit. For primary drying, the shelf temperature is then ramped to −34° C. for 220 minutes and held at −34° C. for 50 hrs. to ensure thorough primary drying. After primary drying, secondary drying is initiated by ramping the shelf temperature up from −34° C. to 25° C. for approximately 9 hrs. and holding at 25° C. for approximately 7 hrs. After the secondary drying step has completed, the cabinet shelf temperature setpoint is reduced to −20° C. until the operator evacuates the cabinet.

For preparation of the PCV24/CL #-LNP lyospheres, the formulations are prepared as described in Example 2, filled into deep-well multichannel pipette plates. 50 μL aliquots of each suspension were dropped onto an ultracold (−180° C.) metal surface (using the “CRYOMEK”) resulting in rapid freezing of 50 μL droplets (A similar process was also done by using a hand pipette and dropping 0.1 mL aliquots onto a liquid nitrogen cooled metal plate). These beads were kept frozen (target −70° C. or below) until they were lyophilized. Lyophilization was performed in a LYOSTAR II unit with a suitable lyophilization cycle.

### Example 5: PCV1 Immunogenicity in Mice: Selection of the Cationic Lipid Formulated as an LNP

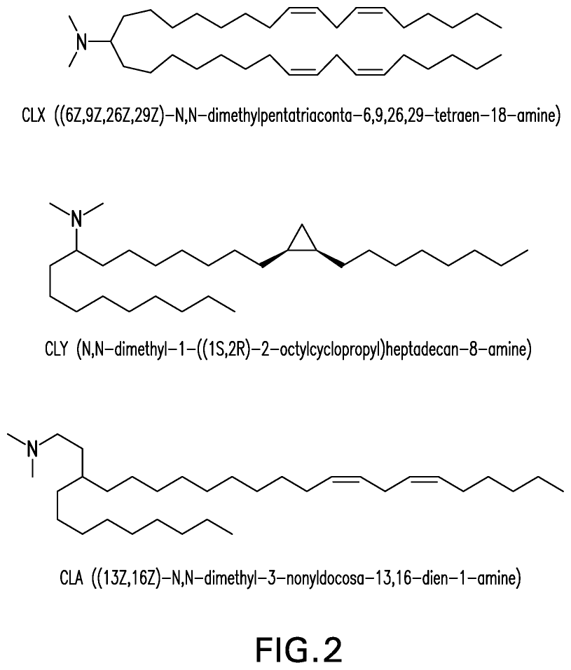

A 1-valent pneumococcal conjugate vaccine (PCV1) using serotype 4 (ST-4) polysaccharide conjugated to CRM197 was prepared as described in Example 2. Three types of cationic lipid containing LNPs were prepared as described in Example 3. CLA-LNP was prepared utilizing the cationic lipid, (13Z,16Z)-N,N-dimethyl-3-nonyldocosa-13,16-dien-1-amine; CLX-LNP was prepared utilizing the cationic lipid, (6Z,9Z,26Z,29Z)-N,N-dimethylpentatriaconta-6,9,26,29-tetraen-18-amine; and CLY-LNP was prepared utilizing the cationic lipid, N,N-dimethyl-1-((1S,2R)-2-octylcyclopropyl)heptadecan-8-amine (FIG. 2). Young female BALB/C mice (6-8 weeks old, n=5/group) were intramuscularly (IM) immunized with 0.1 mL of a 1-valent pneumococcal conjugate vaccine (PCV1) formulated with different cationic containing LNPs based off the cationic lipid concentration on day 0, day 21, and day 42 (Table 2). The pneumococcal conjugate vaccine (ST-4 conjugated to CRM197) was dosed at 0.08 μg PnPs per 0.1 mL immunization. Mice were observed at least daily by trained animal care staff for any signs of illness or distress. The vaccine formulations in mice were deemed to be safe and well tolerated, as no vaccine-related adverse events were noted. All animal experiments were performed in strict accordance with the recommendations in the Guide for Care and Use of Laboratory Animals of the National Institutes of Health. The mouse experimental protocol was approved by the Institutional Animal Care and Use Committee at Merck & Co., Inc.

TABLE 2
Compositions Assessed in PCV1 Immunogenicity Study in Mice
Formulation

ST-4-CRM197; 0.8 μg PnPs/mL; 20 mM Histidine, pH 5.8 and 150 mM NaCl, 0.2% w/v PS-20
[No Adjuvant]
ST-4-CRM197; 0.8 μg PnPs/mL; 20 mM Histidine, pH 5.8 and 150 mM NaCl, 0.2% w/v PS-20
[50 μg/mL Aluminum Phosphate Adjuvant (APA)]
ST-4-CRM197; 0.8 μg PnPs/mL, 10 mM Histidine, pH 5.8 and 150 mM NaCl, with 0.2% w/v
PS-20
[90 μg/mL CLA-LNP Adjuvant]
ST-4-CRM197; 0.8 μg PnPs/mL; 20 mM Histidine, pH 5.8 and 150 mM NaCl, with 0.2% w/v
PS-20
[440 μg/mL CLA-LNP Adjuvant]
ST-4-CRM197; 0.8 μg PnPs/mL; 20 mM Histidine, pH 5.8 and 150 mM NaCl, with 0.2% w/v
PS-20
[2.2 mg/mL CLA-LNP Adjuvant]
ST-4-CRM197; 0.8 μg PnPs/mL; 20 mM Histidine, pH 5.8 and 150 mM NaCl, with 0.2% w/v
PS-20
[2.2 mg/mL CLX-LNP Adjuvant]
ST-4-CRM197; 0.8 μg PnPs/mL; 20 mM Histidine, pH 5.8 and 150 mM NaCl, with 0.2% w/v
PS-20
[440 μg/mL CLY-LNP Adjuvant]
ST-4-CRM197; 0.8 μg PnPs/mL; 20 mM Histidine, pH 5.8 and 150 mM NaCl, with 0.2% w/v
PS-20
2.2 mg/mL CLY- LNP Adjuvant]

Mouse sera were evaluated for IgG immunogenicity using ELISA to assess anti ST-4 IgG titers. PCV1 immunization generated IgG antibody titers in BALB/C mice for the serotype 4 in the vaccine composition (Table 3).

Mice were dosed with various types and concentrations of the cationic lipids (CLA, CLX, or CLY) formulated as LNPs with a consistent dose of a pneumococcal polysaccharide-protein conjugate, serotype 4-CRM197 (referred to as ST-4, or the “serotype 4 antigen” or “antigen”), and their immunogenic response was measured post dose 1 (PD1), post dose 2 (PD2), and post dose 3 (PD3). All formulations of ST-4 prepared with the cationic lipid containing LNPs demonstrated a better enhancement of ST-4 response as compared to ST-4 formulated with or without APA. Further, when comparing ST-4 formulated with the different cationic lipid containing LNPs, LNP formulated with CLA resulted in a better response of ST-4 IgG titer as compared to LNPs formulated with CLX or CLY. CLA dosed at 220 μg with the PCV ST4 antigen resulted in a PD1 Anti-PS4 IgG titer of 42,563. When comparing the PD numbers at the same dose, CLX had a response of 27,280 and CLY had a response of 21,918. After PD3, the response of CLA LNP was 2,947,848 Anti-PS4 IgG, the response from CLX was 1,909,223, and the response from CLY was 1,542,954. All groups with ST-4 formulated with cationic lipid containing LNPs outperformed the ST-4 vaccine formulated with or without APA when dosed into mice.

TABLE 3
Summary of PCV1 Generated Antibody Titers in BALB/C Mice for Serotype 4

| Vaccine Description | Pre | PD1 | PD2 | PD3 |
|---|---|---|---|---|
| ST-4-CRM197; 0.8 μg PnPs/mL | 100 | 100 | 14,888 | 70,322 |
| [No Adjuvant] | | | | |
| ST-4-CRM197; 0.8 μg PnPs/mL | 100 | 3,550 | 189,971 | 430,271 |
| [50 μg/mL APA Adjuvant] | | | | |
| ST-4-CRM197; 0.8 μg PnPs/mL | 100 | 1,390 | 102,466 | 524,890 |
| [90 μg/mL CLA-LNP Adjuvant] | | | | |
| ST-4-CRM197; 0.8 μg PnPs/mL | 100 | 9,311 | 429,802 | 892,967 |
| [440 μg/mL CLA-LNP Adjuvant] | | | | |
| ST-4-CRM197; 0.8 μg PnPs/mL | 100 | 42,563 | 1,473,502 | 2,947,848 |
| [2.2 mg/mL CLA-LNP Adjuvant] | | | | |
| ST-4-CRM197; 0.8 μg PnPs/mL | 100 | 27,280 | 855,319 | 1,909,223 |
| [2.2 mg/mL CLX-LNP Adjuvant] | | | | |
| ST-4-CRM197; 0.8 μg PnPs/mL | 100 | 2,088 | 125,228 | 475,567 |
| [440 μg/mL CLY- LNP Adjuvant] | | | | |
| ST-4-CRM197; 0.8 μg PnPs/mL | 100 | 21,918 | 587,994 | 1,542,954 |
| [2.2 mg/mL CLY- LNP Adjuvant] | | | | |

### Example 6: Immunogenicity of the LNP Adjuvant Comprising CLA in a Multivalent Mouse Study

Young female BALB/C mice (6-8 weeks old, n=10/group) were intramuscularly (IM) immunized with 0.1 mL of a 22-valent pneumiococcal conjugate and polysaccharide vaccine. BALB/C mice were all immunized intramuscularly with 3 doses of 0.1 mL on day 0, day 21, and day 42. In this multivalent mouse study, 22 pneumiococcal polysaccharide-protein conjugates, comprised of the following serotypes: 1, 3, 4, 5, 6A, 6B, 7F, 9V 10A 12F 14, 15A, de-O-acetyl-15B, 18C, 19A, 19F, 22F, 23B, 23F, 24F, 33F, and 35B, each individually conjugated to CRM197, or just the polysaccharides were analyzed for their immune response in the presence of either 1) no adjuvant, 2) APA, and/or 3) an LNP adjuvant formulated using the CLA cationic lipid concentration as an LNP (dose of adjuvant as either Aluminum in the form of APA or CLA as an LNP and dose groups are described in Table 4). Mice were observed at least daily by trained animal care staff for any signs of illness or distress. The vaccine formulations in mice were deemed to be safe and well tolerated, as no vaccine-related adverse events were noted. All animal experiments were performed in strict accordance with the recommendations in the Guide for Care and Use of Laboratory Animals of the National Institutes of Health. The mouse experimental protocol was approved by the Institutional Animal Care and Use Committee at Merck & Co., Inc.

To determine whether the LNP was an effective adjuvant for a PCV, multiple serotypes were tested (Table 4).

TABLE 4
Compositions assessed in PCV22 Immunogenicity Study in Mice
Formulation

PCV22; 0.8 μg PnPs/mL per ST; 20 mM L-histidine, 150 mM NaCl, 0.1% w/v PS-20, pH
5.8 [No Adjuvant]
PCV22; 0.8 μg PnPs/mL per ST; 20 mM L-histidine, 150 mM NaCl, 0.2% w/v PS-20, pH 5.8
[50 μg/mL Aluminum Phosphate Adjuvant (APA)]
PCV22; 0.8 μg PnPs/mL per ST; 20 mM L-histidine, 150 mM NaCl, 0.2% w/v PS-20, pH 5.8
[1250 μg/mL Aluminum Phosphate Adjuvant (APA)]
PCV22; 0.8 μg PnPs/mL per ST; 20 mM L-histidine, 150 mM NaCl, 0.2% w/v PS-20;
[18 μg/mL CLA-LNP Adjuvant]
PCV22; 0.8 μg PnPs/mL per ST; 20 mM L-histidine, 150 mM NaCl, 0.2% w/v PS-20
[440 μg/mL CLA-LNP Adjuvant]
PPSV22; 0.8 μg PnPs/mL per ST; 20 mM L-histidine, 150 mM NaCl, 0.2% w/v PS-20
[440 μg/mL CLA-LNP Adjuvant]

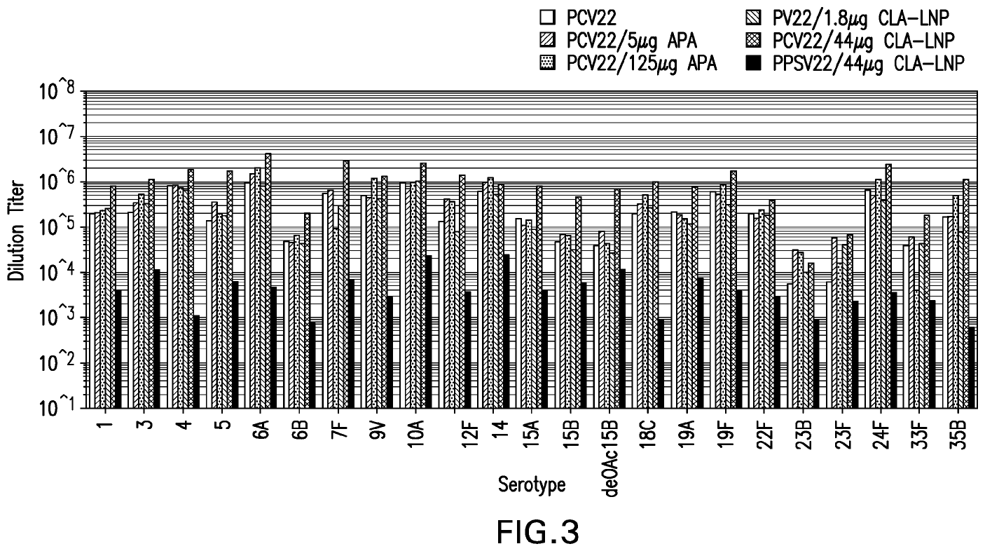

Mouse sera were evaluated for IgG immunogenicity using ECL to assess anti-PCV22 IgG titers, post dose 3 (day 35) (FIG. 3). The PCV22 formulation prepared with the CLA-LNP adjuvant dosed at 44 μg had a comparable or better performance than a PCV22 formulation prepared with APA and dosed at 5 mg for most serotypes after PD1, PD2, and PD3. The results from this study also demonstrate that there is a dose dependent response of CLA in mice. Increasing the concentration of CLA resulted in a higher immunogenic response of PCV22 in mice. When compared to mice dosed with a PCV22 formulation prepared with CLA dosed at 1.8 μg, those dosed with a PCV22 formulated prepared with CLA at 44 μg CLA had a higher immunogenic response for all serotypes post dose 2 (PD2; data not shown) and post dose 3 (PD3 shown in FIG. 3), excluding 10A, 14, 23B, and 23F for both PD2 and PD3 and 22F for PD3 only. A formulation referred to as PPSV22 using unconjugated pneumococcal polysaccharides was prepared as described in Example 2. The results demonstrate that covalent attachment to a carrier protein is required for optimal response when formulated with the CLA-LNP adjuvant.

### Example 7: Immunogenicity Assessment in Infant Rhesus Macaques of a PCV24 Vaccine Prepared with an LNP Adjuvant Comprising CLA

PCV24 (Serotypes-1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B, each individually conjugated to CRM197) and adjuvant formulations were prepared as described in Examples, supra (dose of adjuvant as either aluminum in the form of APA or CLA as an LNP and dose groups are described in Table 5). IRMs (Infant Rhesus Monkeys, n=5/group) were intramuscularly immunized with 100 μL vaccine on days 0, 28 and 56. Sera were collected prior to study start (pre) and on days 42, 70 and 84. IRMs were observed twice daily by trained animal care staff for any signs of illness or distress. The vaccine formulations in IRMs were deemed to be safe and well tolerated, as no vaccine-related adverse events were noted.

TABLE 5
Compositions Assessed in PCV24 Immunogenicity Study in Infant Rhesus Macaques
Formulation

PCV13 (4.4 μg PnPs per Serotype/mL) prepared in 10 mM succinate, 150 mM NaCl,
0.05% w/v PS-80, pH 5.8
[250 μg [Al³⁺]/mL in the form of Aluminum Phosphate]
PCV24 (4.0 μg PnPs per Serotype/mL) prepared in 20 mM L-histidine, 150 mM NaCl,
0.2% w/v PS-20, pH 5.8
[250 μg [Al³⁺]/mL in the form of Aluminum Phosphate Adjuvant (APA)]
PCV24 (4.0 μg PnPs per Serotype/mL) prepared in 10 mM L-histidine, 10 mM Tris, 5%
sucrose, 75 mM NaCl, 0.05% w/v PS-20
[1250 μg/mL CLA-LNP Adjuvant]
PCV24 (4.0 μg PnPs per Serotype/mL) prepared in 10 mM L-histidine, 10 mM Tris, 5%
sucrose, 75 mM NaCl, 0.05% w/v PS-20 Co-lyophilized
[1250 μg/mL CLA-LNP Adjuvant]
PCV24 (4.0 μg PnPs per Serotype/mL) prepared in 10 mM L-histidine, 10 mM Tris, 5%
sucrose, 75 mM NaCl, 0.05% w/v PS-20
[2080 μg/mL CLA-LNP Adjuvant]
PCV24 (4.0 μg PnPs per Serotype/mL) prepared in 10 mM L-histidine, 10 mM Tris, 5%
sucrose, 75 mM NaCl, 0.05% w/v PS-20
[625 μg/mL CLA-LNP Adjuvant & 250 μg[Al³⁺]/mL in the form of Aluminum
Phosphate Adjuvant (APA)]

To assess serotype-specific IgG responses in a 24 valent vaccine, a multiplexed electrochemiluminescence (ECL) assay was developed for use. Endpoint titer was calculated as the reciprocal of the linearly interpolated dilution corresponding to the cutoff value (ECL signal of control) using logarithmic scaling for the ECL and the dilution. Titers were extrapolated for samples beyond the studied maximum dilution, based on linear extrapolation (in the log-log scaling) using the intercept and slope of the last 2 or 3 ECL assay data points for the sample curve completely above the cutoff line. Titers were then obtained by back-transforming the linearly extrapolated dilution. If the sample curve was completely below the cutoff line, 100 was used as the titer in all data analysis and in the Figures.

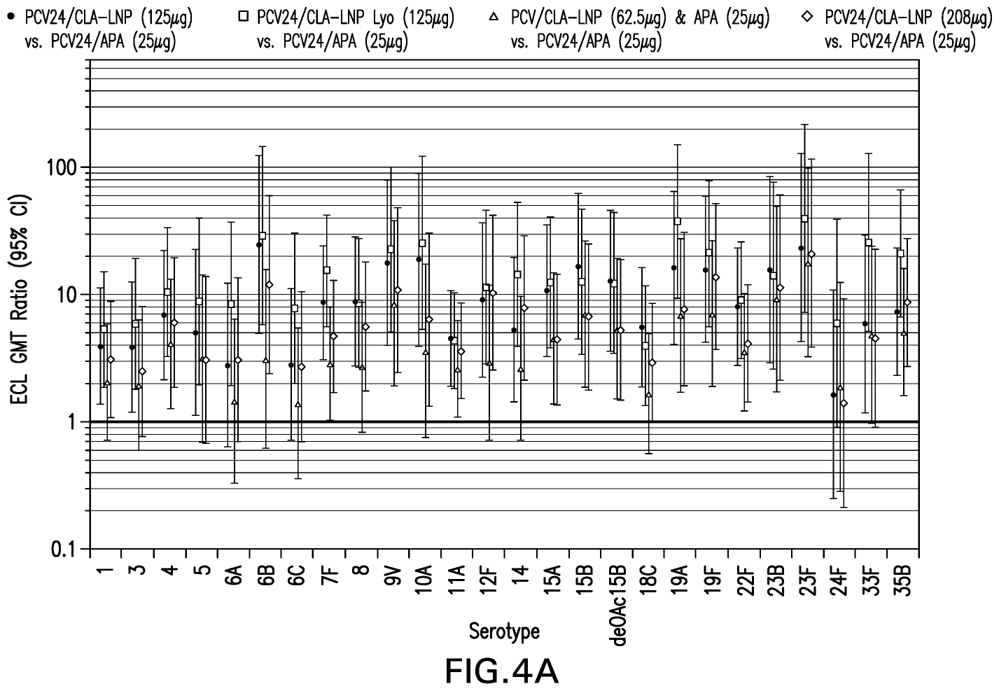

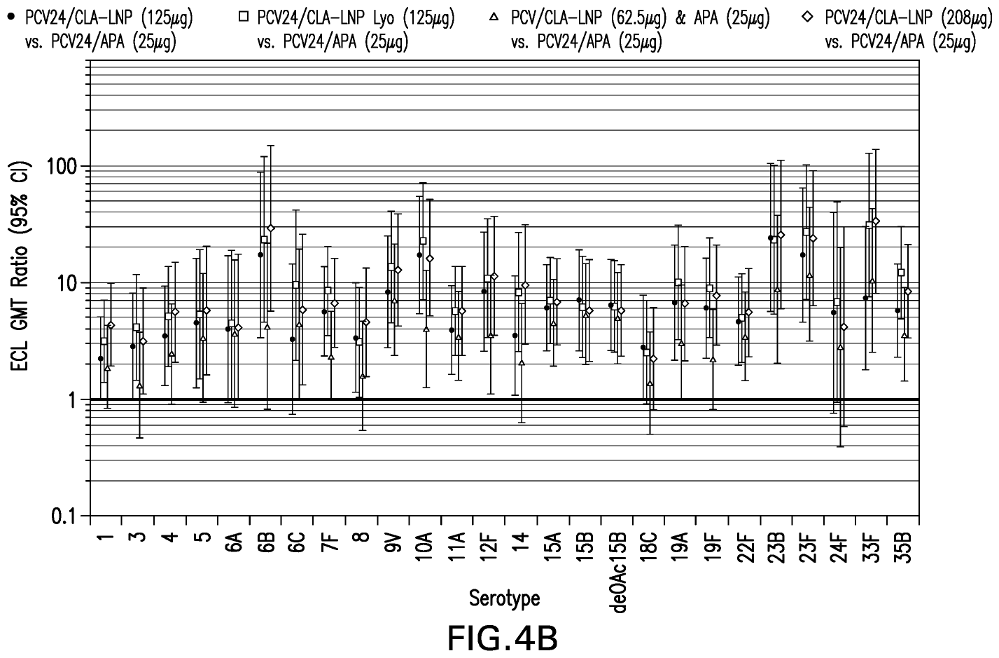

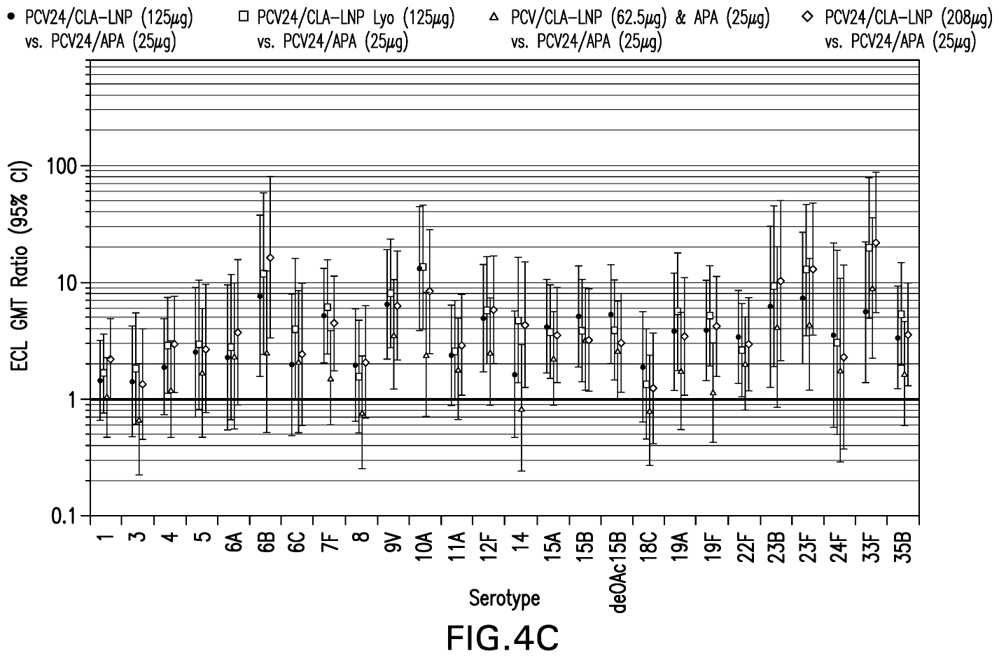

IRM sera were evaluated for IgG immunogenicity using ECL to assess anti-PCV24 IgG titers (FIG. 4A-FIG. 4C). As shown in FIG. 4A, results following post dose 2, measured on day 42, demonstrate that all IRMs dosed with PCV24 formulated with 125 μg CLA-LNP (liquid form and lyophilized form) had a higher or comparable immunogenic response, measured in anti-IgG titers, than those dosed with PCV24 formulated with 25 μg APA (solid line at 1). As shown in FIG. 4B, evaluation of IgG titers after post dose 3, on day 70, demonstrates that all PCV24 serotypes adjuvanted with 125 μg of CLA-LNP (liquid form and lyophilized form) were comparable or better than PCV24 formulated with the APA group (solid line at 1). This comparable or superior response was sustained as evaluated at post dose 3, on day 84 (FIG. 4C).

1-28. (canceled)

29. A composition comprising:
a) at least one Streptococcus pneumoniae polysaccharide; and
b) a lipid nanoparticle (LNP), wherein the LNP comprises a cationic lipid.

30. The composition of claim 29, wherein the at least one Streptococcus pneumoniae polysaccharide is conjugated to a carrier protein.

31. The composition of claim 30, wherein the carrier protein is CRM197.

32. The composition of claim 29, wherein the at least one Streptococcus pneumoniae polysaccharide is selected from the group consisting of serotypes:
4, 6B, 9V, 14, 18C, 19F and 23F;
1, 3, 4, 5, 6A, 6B, 7F, 9V, 14, 18C, 19A, 19F and 23F;
1, 3, 4, 5, 6A, 6B, 7F, 9V, 14, 18C, 19A, 19F, 22F, 23F and 33F;
1, 3, 4, 5, 6A, 6B, 7F, 8, 9V, 10A, 11A, 12F, 14, 15B, 18C, 19A, 19F, 22F, 23F and 33F;
1, 3, 4, 5, 6A, 6B, 7F, 8, 9V, 10A, 11A, 12F, 14, 15A, 15B, 18C, 19A, 19F, 22F, 23B, 23F, 24F, 33F and 35B;
1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B;
1, 3, 4, 5, 6A, 6B, 7F, 8, 9V, 10A, 11A, 12F, 14, 15A, 15C, 18C, 19A, 19F, 22F, 23B, 23F, 24F, 33F and 35B;
3, 6A, 7F, 8, 9N, 10A, 11A, 12F, 15A, 15B, 16F, 17F, 19A, 20A, 22F, 23A, 23B, 24F, 31, 33F and 35B;
3, 6A, 7F, 8, 9N, 10A, 11A, 12F, 15A, de-O-acetylated-15B, 16F, 17F, 19A, 20A, 22F, 23A, 23B, 24F, 31, 33F and 35B;
3, 6A, 7F, 8, 9N, 10A, 11A, 12F, 15A, 15C, 16F, 17F, 19A, 20A, 22F, 23A, 23B, 24F, 31, 33F and 35B;
3, 6A, 7F, 8, 9N, 10A, 11A, 12F, 15A, 15B, 16F, 17F, 19A, 20, 22F, 23A, 23B, 24F, 31, 33F and 35B;
3, 6A, 7F, 8, 9N, 10A, 11A, 12F, 15A, de-O-acetylated-15B, 16F, 17F, 19A, 20, 22F, 23A, 23B, 24F, 31, 33F and 35B;
3, 6A, 7F, 8, 9N, 10A, 11A, 12F, 15A, 15C, 16F, 17F, 19A, 20, 22F, 23A, 23B, 24F, 31, 33F and 35B;
3, 6A, 7F, 8, 9N, 10A, 11A, 12F, 15A, 15B, 16F, 17F, 19A, 20B, 22F, 23A, 23B, 24F, 31, 33F and 35B;
3, 6A, 7F, 8, 9N, 10A, 11A, 12F, 15A, de-O-acetylated-15B, 16F, 17F, 19A, 20B, 22F, 23A, 23B, 24F, 31, 33F and 35B; and
3, 6A, 7F, 8, 9N, 10A, 11A, 12F, 15A, 15C, 16F, 17F, 19A, 20B, 22F, 23A, 23B, 24F, 31, 33F and 35B.

33. The composition of claim 29, wherein the LNP comprises a cationic lipid of Formula 1: 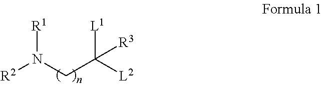
wherein:
R¹ and R² are each methyl;
R³ is H;
n is 1 or 2;
L₁ is selected from C₅-C₂₄ alkyl and C₅-C₂₄ alkenyl; and
L₂ is selected from C₄-C₉ alkyl and C₄-C₉ alkenyl;
or any pharmaceutically acceptable salt or stereoisomer thereof.

34. The composition of claim 29, wherein the LNP comprises a cationic lipid selected from:
DLinDMA;
DLinKC2DMA;
DLin-MC3-DMA;
CLinDMA;
S-Octyl CLinDMA;
(2S)-1-{7-[(3P)-cholest-5-en-3-yloxy]heptyloxy}-3-[(4Z)-dec-4-en-1-yloxy]-N,N-dimethylpropan-2-amine;
(2R)-1-{4-[(3P)-cholest-5-en-3-yloxy]butoxy}-3-[(4Z)-dec-4-en-1-yloxy]-N,N-dimethylpropan-2-amine;
1-[(2R)-1-{4-[(3β)-cholest-5-en-3-yloxy]butoxy}-3-(octyloxy)propan-2-yl]guanidine;
1-[(2R)-1-{7-[(3β)-cholest-5-en-3-yloxy]heptyloxy}-N,N-dimethyl-3-[(9Z,12Z)-octadeca-9,12-dien-1-yloxy]propan-2-amine;
1-[(2R)-1-{4-[(3β)-cholest-5-en-3-yloxy]butoxy}-N,N-dimethyl-3-[(9Z,12Z)-octadeca-9,12-dien-1-yloxy]propan-2-amine;
(2S)-1-({6-[(3P))-cholest-5-en-3-yloxy]hexyl}oxy)-N,N-dimethyl-3-[(9Z)-octadec-9-en-1-yloxy]propan-2-amine;
(3β)-3-[6-{[(2S)-3-[(9Z)-octadec-9-en-1-yloxyl]-2-(pyrrolidin-1-yl)propyl]oxy}hexyl)oxy]cholest-5-ene;
(2R)-1-{4-[(3P)-cholest-5-en-3-yloxy]butoxy}-3-(octyloxy)propan-2-amine;
(2R)-1-({8-[(3P)-cholest-5-en-3-yloxy]octyl)oxy)-N,N-dimethyl-3-(pentyloxy)propan-2-amine;
(2R)-1-({8-[(3P)-cholest-5-en-3-yloxy]octyl}oxy)-3-(heptyloxy)-N,N-dimethylpropan-2-amine;
(2R)-1-({8-[(3P)-cholest-5-en-3-yloxy]octyl}oxy)-N,N-dimethyl-3-[(2Z)-pent-2-en-1-yloxy]propan-2-amine;
(2S)-1-butoxy-3-({8-[(3P)-cholest-5-en-3-yloxy]octyl}oxy)-N,N-dimethylpropan-2-amine;
(2S-1-({8-[(3P)-cholest-5-en-3-yloxy]octyl}oxy)-3-[2,2, 3,3,4,4,5, 5,6, 6,7, 7, 8, 8,9, 9-hexadecafluorononyl)oxy]-N,N-dimethylpropan-2-amine;
2-amino-2-{[(9Z,12Z)-octadeca-9, 12-dien-1-yloxy]methyl} propane-1,3-diol;
2-amino-3-({9-[(3β,8ξ,9ξ,14ξ,17ξ,20ξ)-cholest-5-en-3-yloxy]nonyl}oxy)-2-{[(9Z,12Z)-octadeca-9,12-dien-1-yloxy]methyl}propan-1-ol;
2-amino-3-({6-[(3β,8ξ,9ξ,14ξ,17ξ,20ξ)-cholest-5-en-3-yloxy]nonyl}oxy)-2-{[(9Z)-octadec-9-en-1-yloxy]methyl}propan-1-ol;
(20Z,23Z)-N,N-dimethylnonacosa-20,23-dien-10-amine;
(17Z,20Z)-N,N-dimethylhexacosa-17,20-dien-9-amine;
(16Z,19Z)-N,N-dimethylpentacosa-16,19-dien-8-amine;
(13Z,16Z)-N,N-dimethyldocosa-13,16-dien-5-amine;
(12Z,15Z)-N,N-dimethylhenicosa-12,15-dien-4-amine;
(14Z,17Z)-N,N-dimethyltricosa-14,17-dien-6-amine;
(15Z,18Z)-N,N-dimethyltetracosa-15,18-dien-7-amine;
(18Z,21 Z)-N,N-dimethylheptacosa-18,21-dien-10-amine;
(15Z,18Z)-N,N-dimethyltetracosa-15,18-dien-5-amine;
(14Z,17Z)-N,N-dimethyltricosa-14,17-dien-4-amine;
(19Z,22Z)-N,N-dimethyloctacosa-19,22-dien-9-amine;
(18Z,21Z)-N,N-dimethylheptacosa-18,21-dien-8-amine;
(17Z,20Z)-N,N-dimethylhexacosa-17,20-dien-7-amine;
(16Z,19Z)-N,N-dimethylpentacosa-16,19-dien-6-amine;
(22Z,25Z)-N,N-dimethylhentriaconta-22,25-dien-10-amine;
(21Z,24Z)-N,N-dimethyltriaconta-21,24-dien-9-amine;
(18Z)-N,N-dimethylheptacos-18-en-10-amine;
(17Z)-N,N-dimethylhexacos-17-en-9-amine;
(19Z,22Z)-N,N-dimethyloctacosa-19,22-dien-7-amine;
N,N-dimethylheptacosan-10-amine;
(20Z,23Z)-N-ethyl-N-methylnonacosa-20,23-dien-10-amine;
1-[(11Z,14Z)-1-nonylicosa-11,14-dien-1-yl]pyrrolidine;
(20Z)-N,N-dimethylheptacos-20-en-10-amine;
(15Z)-N,N-dimethylheptacos-15-en-10-amine;
(14Z)-N,N-dimethylnonacos-14-en-10-amine;
(17Z)-N,N-dimethylnonacos-17-en-10-amine;
(24Z)-N,N-dimethyltritriacont-24-en-10-amine;
(20Z)-N,N-dimethylnonacos-20-en-10-amine;
(22Z)-N,N-dimethylhentriacont-22-en-10-amine;
(16Z)-N,N-dimethylpentacos-16-en-8-amine;
(12Z,15Z)-N,N-dimethyl-2-nonylhenicosa-12,15-dien-1-amine;
(13Z,16Z)-N,N-dimethyl-3-nonyldocosa-13,16-dien-1-amine;
N,N-dimethyl-1-[(1S,2R)-2-octylcyclopropyl]heptadecan-8-amine;
1-[(1 S,2R)-2-hexylcyclopropyl]-N,N-dimethylnonadecan-10-amine;
N,N-dimethyl-1-[(1S,2R)-2-octylcyclopropyl]nonadecan-10-amine;
N,N-dimethyl-21-[(1S,2R)-2-octylcyclopropyl]henicosan-10-amine;
N,N-dimethyl-1-[(1S,2S)-2-{[(1R,2R)-2-pentylcyclopropyl]methyl}cyclopropyl]nonadecan-10-amine;
N,N-dimethyl-1-[(1S,2R)-2-octylcyclopropyl]hexadecan-8-amine;
N,N-dimethyl-1-[(1R,2S)-2-undecylcyclopropyl]tetradecan-5-amine;
N,N-dimethyl-3-{7-[(1S,2R)-2-octylcyclopropyl]heptyl}dodecan-1-amine;
1-[(1R,2S)-2-heptylcyclopropyl]-N,N-dimethyloctadecan-9-amine;
1-[(1S,2R)-2-decylcyclopropyl]-N,N-dimethylpentadecan-6-amine;
N,N-dimethyl-1-[(1S,2R)-2-octylcyclopropyl]pentadecan-8-amine; and
(11E,20Z,23Z)-N,N-dimethylnonacosa-11,20,23-trien-10-amine;
or any pharmaceutically acceptable salt or stereoisomer thereof.

35. The composition of claim 29, wherein the LNP comprises a cationic lipid selected from:
(13Z,16Z)-N,N-dimethyl-3-nonyldocosa-13,16-dien-1-amine;
(6Z,9Z,26Z,29Z)-N,N-dimethylpentatriaconta-6,9,26,29-tetraen-18-amine; and
N,N-dimethyl-1-((1S,2R)-2-octylcyclopropyl)heptadecan-8-amine.

36. The composition of claim 29, wherein the LNP is the cationic lipid (13Z,16Z)-N,N-dimethyl-3-nonyldocosa-13,16-dien-1-amine.

37. The composition of claim 29 wherein the LNP further comprises a neutral lipid, a phospholipid, and a PEG-lipid.

38. A composition comprising:
a) at least one Streptococcus pneumoniae polysaccharide-carrier protein conjugate comprising a Streptococcus pneumoniae polysaccharide conjugated to a carrier protein; and
b) a lipid nanoparticle (LNP), wherein the LNP comprises a cationic lipid, a neutral lipid, a phospholipid, and a PEG-lipid;
wherein the at least one Streptococcus pneumoniae polysaccharide is selected from the group consisting of serotypes:
4, 6B, 9V, 14, 18C, 19F and 23F;
1, 3, 4, 5, 6A, 6B, 7F, 9V, 14, 18C, 19A, 19F and 23F;
1, 3, 4, 5, 6A, 6B, 7F, 9V, 14, 18C, 19A, 19F, 22F, 23F and 33F;
1, 3, 4, 5, 6A, 6B, 7F, 8, 9V, 10A, 11A, 12F, 14, 15B, 18C, 19A, 19F, 22F, 23F and 33F;
1, 3, 4, 5, 6A, 6B, 7F, 8, 9V, 10A, 11A, 12F, 14, 15A, 15B, 18C, 19A, 19F, 22F, 23B, 23F, 24F, 33F and 35B;
1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B;
1, 3, 4, 5, 6A, 6B, 7F, 8, 9V, 10A, 11A, 12F, 14, 15A, 15C, 18C, 19A, 19F, 22F, 23B, 23F, 24F, 33F and 35B;
3, 6A, 7F, 8, 9N, 10A, 11A, 12F, 15A, 15B, 16F, 17F, 19A, 20A, 22F, 23A, 23B, 24F, 31, 33F and 35B;
3, 6A, 7F, 8, 9N, 10A, 11A, 12F, 15A, de-O-acetylated-15B, 16F, 17F, 19A, 20A, 22F, 23A, 23B, 24F, 31, 33F and 35B;
3, 6A, 7F, 8, 9N, 10A, 11A, 12F, 15A, 15C, 16F, 17F, 19A, 20A, 22F, 23A, 23B, 24F, 31, 33F and 35B;
3, 6A, 7F, 8, 9N, 10A, 11A, 12F, 15A, 15B, 16F, 17F, 19A, 20, 22F, 23A, 23B, 24F, 31, 33F and 35B;
3, 6A, 7F, 8, 9N, 10A, 11A, 12F, 15A, de-O-acetylated-15B, 16F, 17F, 19A, 20, 22F, 23A, 23B, 24F, 31, 33F and 35B;
3, 6A, 7F, 8, 9N, 10A, 11A, 12F, 15A, 15C, 16F, 17F, 19A, 20, 22F, 23A, 23B, 24F, 31, 33F and 35B;
3, 6A, 7F, 8, 9N, 10A, 11A, 12F, 15A, 15B, 16F, 17F, 19A, 20B, 22F, 23A, 23B, 24F, 31, 33F and 35B;
3, 6A, 7F, 8, 9N, 10A, 11A, 12F, 15A, de-O-acetylated-15B, 16F, 17F, 19A, 20B, 22F, 23A, 23B, 24F, 31, 33F and 35B; and
3, 6A, 7F, 8, 9N, 10A, 11A, 12F, 15A, 15C, 16F, 17F, 19A, 20B, 22F, 23A, 23B, 24F, 31, 33F and 35B;
and the carrier protein is CRM197.

39. The composition of claim 38, wherein the cationic lipid is selected from:
(13Z,16Z)-N,N-dimethyl-3-nonyldocosa-13,16-dien-1-amine;
(6Z,9Z,26Z,29Z)-N,N-dimethylpentatriaconta-6,9,26,29-tetraen-18-amine; and
N,N-dimethyl-1-((1S,2R)-2-octylcyclopropyl)heptadecan-8-amine.

40. The composition of claim 38, wherein the cationic lipid is (13Z,16Z)-N,N-dimethyl-3-nonyldocosa-13,16-dien-1-amine.

41. A composition comprising:
a) at least one Streptococcus pneumoniae polysaccharide-carrier protein conjugate comprising a Streptococcus pneumoniae polysaccharide conjugated to a carrier protein; and
b) a lipid nanoparticle (LNP), wherein the LNP comprises a cationic lipid, cholesterol, ePEG2000-DMG, and distearoyl phosphatidyl choline (DSPC).

42. The composition of claim 41, wherein the LNP comprises a cationic lipid of Formula 1: 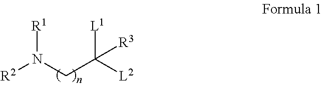
wherein:
R¹ and R² are each methyl;
R³ is H;
n is 1 or 2;
L₁ is selected from C₅-C₂₄ alkyl and C₅-C₂₄ alkenyl; and
L₂ is selected from C₄-C₉ alkyl and C₄-C₉ alkenyl;
or any pharmaceutically acceptable salt or stereoisomer thereof.

43. The composition of claim 41, wherein the LNP comprises a cationic lipid selected from:
DLinDMA;
DLinKC2DMA;
DLin-MC3-DMA;
CLinDMA;
S-Octyl CLinDMA;
(2S)-1-{7-[(3P)-cholest-5-en-3-yloxy]heptyloxy}-3-[(4Z)-dec-4-en-1-yloxy]-N,N-dimethylpropan-2-amine;
(2R)-1-{4-[(3P)-cholest-5-en-3-yloxy]butoxy}-3-[(4Z)-dec-4-en-1-yloxy]-N,N-dimethylpropan-2-amine;
1-[(2R)-1-{4-[(3β)-cholest-5-en-3-yloxy]butoxy}-3-(octyloxy)propan-2-yl]guanidine;
1-[(2R)-1-{7-[(3β)-cholest-5-en-3-yloxy]heptyloxy}-N,N-dimethyl-3-[(9Z,12Z)-octadeca-9,12-dien-1-yloxy]propan-2-amine;
1-[(2R)-1-{4-[(3β)-cholest-5-en-3-yloxy]butoxy}-N,N-dimethyl-3-[(9Z,12Z)-octadeca-9,12-dien-1-yloxy]propan-2-amine;
(2S)-1-({6-[(3P))-cholest-5-en-3-yloxy]hexyl}oxy)-N,N-dimethyl-3-[(9Z)-octadec-9-en-1-yloxy]propan-2-amine;
(3β)-3-[6-{[(2S)-3-[(9Z)-octadec-9-en-1-yloxyl]-2-(pyrrolidin-1-yl)propyl]oxy}hexyl)oxy]cholest-5-ene;
(2R)-1-{4-[(3P)-cholest-5-en-3-yloxy]butoxy}-3-(octyloxy)propan-2-amine;
(2R)-1-({8-[(3P)-cholest-5-en-3-yloxy]octyl)oxy)-N,N-dimethyl-3-(pentyloxy)propan-2-amine;
(2R)-1-({8-[(3P)-cholest-5-en-3-yloxy]octyl}oxy)-3-(heptyloxy)-N,N-dimethylpropan-2-amine;
(2R)-1-({8-[(3P)-cholest-5-en-3-yloxy]octyl}oxy)-N,N-dimethyl-3-[(2Z)-pent-2-en-1-yloxy]propan-2-amine;
(2S)-1-butoxy-3-({8-[(3P)-cholest-5-en-3-yloxy]octyl}oxy)-N,N-dimethylpropan-2-amine;
(2S-1-({8-[(3P)-cholest-5-en-3-yloxy]octyl}oxy)-3-[2,2, 3,3,4,4,5, 5,6, 6,7, 7, 8, 8,9, 9-hexadecafluorononyl)oxy]-N,N-dimethylpropan-2-amine;
2-amino-2-{[(9Z,12Z)-octadeca-9, 12-dien-1-yloxy]methyl} propane-1,3-diol;
2-amino-3-({9-[(3ξ,8ξ,9ξ,14ξ,17ξ,20ξ)-cholest-5-en-3-yloxy]nonyl}oxy)-2-{[(9Z,12Z)-octadeca-9,12-dien-1-yloxy]methyl}propan-1-ol;
2-amino-3-({6-[(3β,8ξ,9ξ,14ξ,17ξ,20ξ)-cholest-5-en-3-yloxy]nonyl}oxy)-2-{[(9Z)-octadec-9-en-1-yloxy]methyl}propan-1-ol;
(20Z,23Z)-N,N-dimethylnonacosa-20,23-dien-10-amine;
(17Z,20Z)-N,N-dimethylhexacosa-17,20-dien-9-amine;
(16Z,19Z)-N,N-dimethylpentacosa-16,19-dien-8-amine;
(13Z,16Z)-N,N-dimethyldocosa-13,16-dien-5-amine;
(12Z,15Z)-N,N-dimethylhenicosa-12,15-dien-4-amine;
(14Z,17Z)-N,N-dimethyltricosa-14,17-dien-6-amine;
(15Z,18Z)-N,N-dimethyltetracosa-15,18-dien-7-amine;
(18Z,21 Z)-N,N-dimethylheptacosa-18,21-dien-10-amine;
(15Z,18Z)-N,N-dimethyltetracosa-15,18-dien-5-amine;
(14Z,17Z)-N,N-dimethyltricosa-14,17-dien-4-amine;
(19Z,22Z)-N,N-dimethyloctacosa-19,22-dien-9-amine;
(18Z,21Z)-N,N-dimethylheptacosa-18,21-dien-8-amine;
(17Z,20Z)-N,N-dimethylhexacosa-17,20-dien-7-amine;
(16Z,19Z)-N,N-dimethylpentacosa-16,19-dien-6-amine;
(22Z,25Z)-N,N-dimethylhentriaconta-22,25-dien-10-amine;
(21 Z,24Z)-N,N-dimethyltriaconta-21,24-dien-9-amine;
(18Z)-N,N-dimethylheptacos-18-en-10-amine;
(17Z)-N,N-dimethylhexacos-17-en-9-amine;
(19Z,22Z)-N,N-dimethyloctacosa-19,22-dien-7-amine;
N,N-dimethylheptacosan-10-amine;
(20Z,23Z)-N-ethyl-N-methylnonacosa-20,23-dien-10-amine;
1-[(11Z,14Z)-1-nonylicosa-11,14-dien-1-yl]pyrrolidine;
(20Z)-N,N-dimethylheptacos-20-en-10-amine;
(15Z)-N,N-dimethylheptacos-15-en-10-amine;
(14Z)-N,N-dimethylnonacos-14-en-10-amine;
(17Z)-N,N-dimethylnonacos-17-en-10-amine;
(24Z)-N,N-dimethyltritriacont-24-en-10-amine;
(20Z)-N,N-dimethylnonacos-20-en-10-amine;
(22Z)-N,N-dimethylhentriacont-22-en-10-amine;
(16Z)-N,N-dimethylpentacos-16-en-8-amine;
(12Z,15Z)-N,N-dimethyl-2-nonylhenicosa-12,15-dien-1-amine;
(13Z,16Z)-N,N-dimethyl-3-nonyldocosa-13,16-dien-1-amine;
N,N-dimethyl-1-[(1S,2R)-2-octylcyclopropyl]heptadecan-8-amine;
1-[(1 S,2R)-2-hexylcyclopropyl]-N,N-dimethylnonadecan-10-amine;
N,N-dimethyl-1-[(1S,2R)-2-octylcyclopropyl]nonadecan-10-amine;
N,N-dimethyl-21-[(1S,2R)-2-octylcyclopropyl]henicosan-10-amine;
N,N-dimethyl-1-[(1S,2S)-2-{[(1R,2R)-2-pentylcyclopropyl]methyl}cyclopropyl]nonadecan-10-amine;
N,N-dimethyl-1-[(1S,2R)-2-octylcyclopropyl]hexadecan-8-amine;
N,N-dimethyl-1-[(1R,2S)-2-undecylcyclopropyl]tetradecan-5-amine;
N,N-dimethyl-3-{7-[(1 S,2R)-2-octylcyclopropyl]heptyl}dodecan-1-amine;
1-[(1R,2S)-2-heptylcyclopropyl]-N,N-dimethyloctadecan-9-amine;
1-[(1S,2R)-2-decylcyclopropyl]-N,N-dimethylpentadecan-6-amine;
N,N-dimethyl-1-[(1S,2R)-2-octylcyclopropyl]pentadecan-8-amine; and
(11E,20Z,23Z)-N,N-dimethylnonacosa-11,20,23-trien-10-amine;
or any pharmaceutically acceptable salt or stereoisomer thereof.

44. The composition of claim 41, wherein the LNP comprises a cationic lipid selected from:
(13Z,16Z)-N,N-dimethyl-3-nonyldocosa-13,16-dien-1-amine;
(6Z,9Z,26Z,29Z)-N,N-dimethylpentatriaconta-6,9,26,29-tetraen-18-amine; and
N,N-dimethyl-1-((1S,2R)-2-octylcyclopropyl)heptadecan-8-amine.

45. The composition of claim 41, wherein the LNP comprises the cationic lipid (13Z,16Z)-N,N-dimethyl-3-nonyldocosa-13,16-dien-1-amine.

46. A composition comprising:
a) at least one Streptococcus pneumoniae polysaccharide-carrier protein conjugate comprising a Streptococcus pneumoniae polysaccharide conjugated to a carrier protein; and
b) a lipid nanoparticle (LNP), wherein the LNP comprises a cationic lipid, cholesterol, ePEG2000-DMG, and distearoyl phosphatidyl choline (DSPC);
wherein the at least one Streptococcus pneumoniae polysaccharide is selected from the group consisting of serotypes:
3, 6A, 7F, 8, 9N, 10A, 11A, 12F, 15A, 15B, 16F, 17F, 19A, 20B, 22F, 23A, 23B, 24F, 31, 33F and 35B;
3, 6A, 7F, 8, 9N, 10A, 11A, 12F, 15A, de-O-acetylated-15B, 16F, 17F, 19A, 20B, 22F, 23A, 23B, 24F, 31, 33F and 35B; and
3, 6A, 7F, 8, 9N, 10A, 11A, 12F, 15A, 15C, 16F, 17F, 19A, 20B, 22F, 23A, 23B, 24F, 31, 33F and 35B;
wherein the carrier protein is CRM197; and
wherein the cationic lipid selected from:
(13Z,16Z)-N,N-dimethyl-3-nonyldocosa-13,16-dien-1-amine;
(6Z,9Z,26Z,29Z)-N,N-dimethylpentatriaconta-6,9,26,29-tetraen-18-amine; and
N,N-dimethyl-1-((1S,2R)-2-octylcyclopropyl)heptadecan-8-amine.

47. A composition comprising:
a) at least one Streptococcus pneumoniae polysaccharide-carrier protein conjugate comprising a Streptococcus pneumoniae polysaccharide conjugated to a carrier protein; and
b) a lipid nanoparticle (LNP), wherein the LNP comprises a cationic lipid, cholesterol, ePEG2000-DMG, and distearoyl phosphatidyl choline (DSPC);
wherein the at least one Streptococcus pneumoniae polysaccharide is selected from the group consisting of serotypes:
3, 6A, 7F, 8, 9N, 10A, 11A, 12F, 15A, 15B, 16F, 17F, 19A, 20B, 22F, 23A, 23B, 24F, 31, 33F and 35B;
3, 6A, 7F, 8, 9N, 10A, 11A, 12F, 15A, de-O-acetylated-15B, 16F, 17F, 19A, 20B, 22F, 23A, 23B, 24F, 31, 33F and 35B; and
3, 6A, 7F, 8, 9N, 10A, 11A, 12F, 15A, 15C, 16F, 17F, 19A, 20B, 22F, 23A, 23B, 24F, 31, 33F and 35B;
wherein the carrier protein is CRM197; and
wherein the cationic lipid is (13Z,16Z)-N,N-dimethyl-3-nonyldocosa-13,16-dien-1-amine.
